SEQUENCE LISTING (22 sequences)

SEQ ID NO: 1 - protein, 95 aa, Pichinde virus
MGLRYSKEVRKRHGDEDVVGRVPMTLNLPQGLYGRFNCKSCWFVNKGLIRCKDHYLCLGCLTKMHSRGNLCEICGHSLPTKMEFLESPSAPPYEP

SEQ ID NO: 2 - protein, 2194 aa, Pichinde virus
MEEYVFELKDIVRKWVPEWEELSEQKNNVLAQVKDRAITIEGLKLLSMLVEVDSCKKHSCKHNTKMTVNAILRELRVTCPTLPDVTPDGYCMVGDVLILLEVFVRTSQEAFEKKYNQDFLKLLQLSSDLKRQNITLVPVIDGRSSYYVEFVPDWVVERLRWLLLKLMDGLRTSGEEVEELEYERLISSLSSLENQSLGLESLLAVKERGLPYKVRLEKALMSGINNKLTTDQCRTKIMEIFQQFKMLQLAGQLDRKLQATDREDMISRLQNHEFIQCSVKDVPKSEIRLCEFCSVHILGIIGQLRQSEVKHSSTESREYFRVLSICNKIKSQKVFNTRRNTMLVLDLIMYNILCDLDKSSPGAVFREVLLMQGLPSVNDRLINVDFLMEQITKKFIKNPNWLEKAKKRLSSVCGELPLDDILPLLREPDVEYYFNLKTSVLDEWGAKPCLQYKTKSQCMCGGRPGRGQPDYTIMGESEFEELLKTLSSLSLSLINSMKTAAVPKMKVNNADEFYGKVYCDEVFFQRFGEGGSLTLLYQKTGERSRCYAVAYRSKSGGLYETKASFYCDPKRFFLPIFSADVIQRTCVEMLSWLDFMSQPLLDSVSDLLRRLILCILCTPSKRIQVYLQGFRYYIMAFVNEVHFKELFEKLKVVMLTPSEWQTAMLIDDLILLVLSNSREEDMAKIFKFVLNVSYLCHFITKETPDRLTDQIKCFEKFLEPKLKFDSVLVNPSNSMELPTEEEEKMVHDIERLLGKKLESKCEGRPGLNKDVLSVCLSLFNSSSLEVKPLLPCDPMTPSFTSTALDMSSNKSVVVPKLNEVGEVITEYDYSSIVSAVVVEMIEHFKTKGKYKLDPKEVNFKILKRLSSLIQIKKESIEPDGVEELLSEDQGDCLKEIETRVAKVLSKVDTNVKTNLKTSCPLERLWPKSTMVVIKRETSLHDVKDFDYSLFSAEVYEDLVNLIYEDVTARSVYFADRLMNPCPLEFLIKNLTLKAYKEADYFECFKYILIASDYDNRVGRYDHKSRSRLGFTDAALQIRETSRISSRESNSESIAKRLDQSFFTNSSLRNLCFYSDESPTERSGVSTNVGRLKFGLSYKEQVGGNRELYVGDLNTKLTTRLIEDYSESLMQNMRYTCLNNEKEFERALLDMKSVVRQSGLAVSMDHSKWGPHMSPVIFAALLKGLEFKLKDGSEVPNAAVINILLWHIHKMVEVPFNVVEAYMKGFLKRGLGMMDKGGCTIAEEFMFGYFEKGKVPSHISSVLDMGQGILHNTSDLYGLITEQFINYALELCYGARFISYTSSDDEIMLSLNEGFKFKDRDELNVELVLDCMEFHYFLSDKLNKFVSPKTVVGTFASEFKSRFFIWSQEVPLLTKFVAAALHNIKAKAPNQQADTIDTILDQCVANGVSIEVVGAIAKRTNSMIIYSGFPNDPFLCLEEMDVLDWVNGSRGYRLQRSIETLFPDDLLLSIIRKACRKIFYKIQSGALEESYIVTTLQQSPDDCLKQLLETCDVETEAIEDALNIRWLNLRVHGDLRLVLRTKLMSTTRTVQREEIPSLVKSVQSKLSKNYVRGAKKILADAINKSAFQSSIASGFIGVCKSMGSKCVRDGKGGFKYIRDITSKIILHRDCHFCNQRKGVYCKAALGEVSEYSRPLIWDYFALVLTNACELGNWVFQKAEVPKIVTHLNNPNHFWPIKPSTHSELEDKVGINHILYSIRRNFPTLFDEHISPFLSDLNMLRLSWVQRIKFLDLCVAIDITSECLGIVSHIIKHRREELYIVKQNELAMSHSRESHPLERGFNLEPEEVCTNFLIQILFESMLVPVIMSTSQFKKYFWFGELELLPNNAQHDLKQLTQFICDCKKNNTSRTMNLDDLDVGFVSSKLILSCVNLNISVFINELDWVNRDNYENIEQLILASPSEVIPIELNLTFSHKRVSHKFRYERSTNYILKLRFLIERESLLDSLDSDGYLLLNPHSVEYYVSQSSGNHISLDGVSLLVLNPLINGKDVLDFNDLLEGQDIHFKSRSTVFQKVRIDLKNRFKDLKNKFSYKLIGPDVGMQPLILEGGLIKEGNRVVSRLEVNLDSKVVIIALEALEPEKRPRFIANLFQYLSSAQSHNKGISMNEQDLRLMIENFPEVFEHMLHDAKDWLNCGHFSIIRSKTLGSVMIADETGPFKIKGIRCRKLFEDNESVEIE

SEQ ID NO: 3 - protein, 561 aa, Pichinde virus
MSDNIPSFRWVQSLRRGLSNWTHPVKADVLSDTRALLSALDFHKVAQVQRMMRKDKRTDSDLTKLRDMNKEVDALMNMRSIQRDNVLKVGGLAKEELMELASDLDKLRKKVTRTESLSQPGVYGGNLTNTQLEQRAEILRSMGFANARPTGNRDGVVKIWDIKDNTLLINQFGSMPALTIACMTEQGGEQLNDVVQALSALGLLYTVKFPNMTDLEKLTQQHSALKIISNEPSAINISGYNLSLSAAVKAAACMIDGGNMLETIQVKPSMFSTLIKSLLQIKNREGMFVSTTPGQRNPYENLLYKICLSGDGWPYIGSRSQVQGRAWDNTTVDLDSKPSAIQPPVRNGGSPDLKQIPKEKEDTVVSSIQMLDSKATTWIDIEGTPNDPVEMAIYQPDTGNYIHCYRFPHDEKSFKEQSKYSHGLLLKDLADAQPGLISSIIRHLPQNMVFTAQGSDDIISLFEMHGRRDLKVLDVKLSAEQARTFEDEIWERYNLLCTKHKGLVIKKKKKGAAQTTANPHCALLDTIMFDATVTGWVRDQKPMRCLPIDTLYRNNTDLINL

SEQ ID NO: 4 - protein, 508 aa, Pichinde virus
MGQVVTLIQSIPEVLQEVFNVALIIVSTLCIIKGFVNLMRCGLFQLITFLILAGRSCDGMMIDRRHNLTHVEFNLTRMFDNLPQSCSKNNTHHYYKGPSNTTWGIELTLTNTSIANETTGNFSNIRSLAYGNISNCDKTEEAGHTLKWLLNELHFNVLHVTRHVGARCKTVEGAGVLIQYNLTVGDRGGEVGRHLIASLAQIIGDPKIAWVGKCFNNCSGGSCRLTNCEGGTHYNFLIIQNTTWENHCTYTPMATIRMALQKTAYSSVSRKLLGFFTWDLSDSTGQHVPGGYCLEQWAIVWAGIKCFDNTVMAKCNKDHNEEFCDTMRLFDFNQNAIKTLQLNVENSLNLFKKTINGLISDSLVIRNSLKQLAKIPYCNYTKFWYINDTITGRHSLPQCWLVHNGSYLNETHFKNDWLWESQNLYNEMLIKEYEERQGKTPLALTDICFWSLVFYTITVFLHLVGIPTHRHIIGDGCPKPHRITRNSLCSCGYYKIPKKPYKWVRLGK

SEQ ID NO: 5 - protein, 325 aa, Mycobacterium tuberculosis
MTDVSRKIRAWGRRLMIGTAAAVVLPGLVGLAGGAATAGAFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAG

SEQ ID NO: 6 - protein, 95 aa, Mycobacterium tuberculosis
MTEQQWNFAGIEAAASAIQGNVTSIHSLLDEGKQSLTKLAAAWGGSGSEAYQGVQQKWDATATELNNALQNLARTISEAGQAMASTEGNVTGMFA

SEQ ID NO: 7 - protein, 96 aa, Mycobacterium tuberculosis
MSQIMYNYPAMLGHAGDMAGYAGTLQSLGAEIAVEQAALQSAWQGDTGITYQAWQAQWNQAMEDLVRAYHAMSSTHEANTMAMMARDTAEAAKWGG

SEQ ID NO: 8 - protein, 21 aa, synthetic construct
GSGEGRGSLLTCGDVEENPGP

SEQ ID NO: 9 - protein, 22 aa, synthetic construct
GSGATNFSLLKQAGDVEENPGP

SEQ ID NO: 10 - protein, 23 aa, synthetic construct
GSGQCTNYALLKLAGDVESNPGP

SEQ ID NO: 11 - protein, 25 aa, synthetic construct
GSGVKQTLNFDLLKLAGDVESNPGP

SEQ ID NO: 12 - RNA, 84 nt, Pichinde virus
  source 1..84: mol_type = other RNA
cgcaccggggatcctaggcatctttgggtcacgcttcaaatttgtccaatttgaacccagctcaagtcctggtcaaaacttggg

SEQ ID NO: 13 - RNA, 30 nt, Pichinde virus
  source 1..30: mol_type = genomic RNA
cgcaccgaggatcctaggcatttcttgatc

SEQ ID NO: 14 - RNA, 51 nt, Pichinde virus
  source 1..51: mol_type = genomic RNA
cgcaccggggatcctaggcataccttggacgcgcatattacttgatcaaag

SEQ ID NO: 15 - RNA, 83 nt, Pichinde virus
  source 1..83: mol_type = genomic RNA
cgcacagtggatcctaggcgattctagatcacgctgtacgttcacttcttcactgactcggaggaagtgcaaacaaccccaaa

SEQ ID NO: 16 - RNA, 71 nt, Pichinde virus
  source 1..71: mol_type = genomic RNA
accagggcccctgggcgcacccccctccgggggtgcgcccgggggcccccggccccatggggccggttgtt

SEQ ID NO: 17 - RNA, 75 nt, Pichinde virus
  source 1..75: mol_type = genomic RNA
gccctagcctcgacatgggcctcgacgtcactccccaataggggagtgacgtcgaggcctctgaggacttgagct

SEQ ID NO: 18 - RNA, 87 nt, Pichinde virus
  source 1..87: mol_type = genomic RNA
agctctcccttagccatccgagtggacgacgtcctccttcggatgcccaggtcggaccgcgaggaggtggagatgccatgccgaccc

SEQ ID NO: 19 - DNA, 1608 bp, synthetic construct
  source 1..1608: mol_type = other DNA
gaattccaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtacaacgcagagctccgtccaacatgactacagcgagagatattatgaatgctggagtgacttgcgtcggagagcatgagactctgacagccgcagcacagtatatgcgggagcacgatatcggagccctcccaatttgtggtgacgacgatcgccttcatgggatgctgactgatagggatattgtaatcaaggggctggctgcaggcctggatcccaacacagccactgccggcgagctggctagggattctatttactacgtggacgctaacgcatccatccaagagatgctgaatgtgatggaggaacaccaggtgcggagggtgccagtgatctctgaacatcggctcgtaggaatcgtcacggaggccgatatcgctcggcatctgcccgaacacgcaatagtacagttcgtgaaagcaatctgctcccccatggctctggcaagcatgatagccaccaccagggacagagaaggcgctacgatgatcacgttcagactgaggctgccatgtagaacaatcctgcgggtgttttccaggaacccattggttagagggactgaccggttggaagccgtcgtcatgctgcttgccgtcacagtcagcctgctgaccattccttttgccgctgctgccgggaccgcggtgcaggacagccggtcacatgtatacgcccaccaggcccagaccagacaccctgcaaccgccaccgtaatcgatcacgaaggggtcatcgactctaacaccactgctacatcagctcctcctagaacaaagattacggttcctgcgaggtgggtcgtgaatggcatcgaacggagtggggaagtgaacgccaaacctggcactaaatccggtgatcgcgtcggaatttgggtggactcagccggccagttggtcgatgagcctgccccacccgctcgcgcaatcgccgacgcggccctggcagcactcggtttgtggctttccgtagcggccgttgccggcgcattgctggctctgacacgcgctatcctcatacgagtgaggaatgcctcttggcaacatgatatagacagcctcttttgtacccagataataggcggccgcagatcttacgtagaagttcctattctctagaaagtataggaacttccatatggctagaagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatg

SEQ ID NO: 20 - DNA, 2757 bp, synthetic construct
  source 1..2757: mol_type = other DNA
gaattccaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtacaacgcagagctcgtcaacatgtcagggaggcatcggaaaccaactacaagcaatgtatctgttgccaagattgctttcaccggcgcagttcttggaggtggcggaattgccatggctgcccaggcaacagccgctacagatggagagtgggatcaggtggctcgatgtgagtctggtggcaactggtctatcaacactgggaacgggtatcttggcggcttgcaatttactcagagcacttgggctgcccacggagggggtgaatttgctcctagcgcgcagctggcctcccgcgagcagcagatcgctgtgggagagagggtgttggccacacagggaagaggtgcctggcctgtctgtggccgcggactcagtaatgctacccctagggaggtgctgcccgcctcagccgctatggacgctccactggatgctgccgccgtgaatggcgagccagctccgctggcacccccacctgcagaccccgctcccccagtcgagctggcggcaaacgacctgcccgcacctctcggagaaccacttcctgcagcgcctgccgatccagctccacctgctgatttggctccccccgctcccgccgatgtagcccctccggtcgagttggctgtgaatgacctgccggcacctctgggcgagcccctcccagccgctccggccgaccctgcccctcctgctgatctggcaccacccgctcctgccgacctcgccccacccgccccagcagacctggctccaccagcgcctgcggatcttgccccgcctgttgagctggctgtcaacgatcttcctgcgcctcttggagagcccctgcccgctgctccagccgaactcgcaccaccggcagatctggctcccgcctctgccgatcttgcacctcccgcaccggcggacttggcacctccagcaccagcagaactggctccccctgcgccggctgacctggcccctccagcagccgttaatgagcaaaccgcaccaggggaccagccggctacggcaccaggtggaccggtggggctggccaccgacctggagctgcctgagccggatccccaaccagctgatgctcccccacctggcgacgtaactgaggccccagctgaaacgccccaggtcagtaacatcgcttacacaaagaaactgtggcaggcaattagggctcaggacgtgtgtgggaacgacgccctggacagcttggcccaaccgtacgtgatcggtatgcaccccctccccgctgatcatggtcgcagtcgctgtaaccgccaccccatttcacctctcagccttattgggaatgcgtctgctacaagtggcgacatgtctagtatgacaaggattgctaagcccctcatcaaaagtgcgatggctgccggtctggtaacagcatccatgagcttgtccaccgcagtggctcacgctgggccttccccgaactgggatgccgtcgcccagtgcgagtcaggcggcaattgggccgcaaataccggtaacggtaagtatggaggactgcagtttaaacctgcaacttgggccgcctttggaggagtgggtaatcctgcagctgcttctagagaacagcagattgccgtggctaaccgcgttctcgcggagcagggtctggacgcctggccgacctgtggcgccgcatcaggtttgccgatcgcgttgtggtcaaagcccgcccagggaatcaagcagattatcaatgagatcatctgggccggaatacaggcaagcatccctagaatgactcctgggcttctgacaaccgctggcgctgggaggcccagggataggtgcgcccggatcgtttgtaccgtattcatagagaccgccgtggtcgcgacaatgttcgtggctctcttgggcttgagcaccattagctctaaggccgatgatatagattgggatgctattgctcaatgcgaatccggtgggaactgggccgctaataccggaaatgggctctacggcggactgcagatcagccaggctacatgggatagcaacggaggagtcgggtcccctgccgctgcatccccgcaacagcaaatcgaggtggccgataacatcatgaaaacccagggacccggagcctggcccaaatgtagctcatgtagccaaggagatgcgcccctcggttcactgacgcacatcctcaccttcctcgccgcggaaaccggagggtgctctggcagccgggacgactgagcggccgcagatcttacgtagaagttcctattctctagaaagtataggaacttccatatggctagaagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatg

SEQ ID NO: 21 - RNA, 7057 nt, Pichinde virus
  source 1..7057: mol_type = genomic RNA
cgcaccggggatcctaggcatctttgggtcacgcttcaaatttgtccaatttgaacccagctcaagtcctggtcaaaacttgggatgggactcagatatagcaaagaggtcaggaagagacatggcgacgaagatgtggtgggaagggtccccatgaccctcaatctaccacagggcctgtatggcaggttcaactgcaaatcttgctggttcgtcaacaaaggtctcatcaggtgcaaagaccactatctgtgtcttgggtgcttaaccaaaatgcactccagaggcaatctctgcgagatatgcggccactcactgccaaccaagatggagttcctagaaagcccctctgcaccaccctacgagccataaaccagggcccctgggcgcacccccctccgggggtgcgcccgggggcccccggccccatggggccggttgtttactcgatctccactgactcattgtcctcaaacaactttcgacacctgattcccttgatcttgaagggtcctgtctcgtctgcaatcataacagatcctagagtcttacttcttattatactaaagtgaccacaattcaaccaatctttggcatcatgcaacatgtgttcaaacacttcggggaaattttcaatcatgagtcttaaatcctgctcgttcatacttattcccttgttgtgagactgtgcacttgaaaggtactgaaaaaggttggcaataaatcttggccttttctcaggttctaatgcttccagtgcaatgatgaccacctttgagtctaagttcacttccaatctagaaaccactctgttgccctctttgatcaacccaccctctaaaatgaggggttgcatcccaacatcaggaccaatcaacttataggaaaatttgtttttcaaatccttgaaacgatttttcaaatctattctcaccttctggaacacagttgaccttgacttgaagtgaatgtcttgaccttccaatagatcattgaagtctagaacatcttttccgttgatgagaggattcagaaccaaaagtgacacaccatccagacttatgtgattcccggaagattgagaaacataatactcaacagaatgggggttcaacaataggtaaccatcagagtccaatgagtccagcaatgactccctttcaataagaaatcttaattttaatatgtaattggtagacctctcatatctaaatttgtggctcactctcttatgagaaaatgttaggttgagctcaatgggaatgacctcagaaggtgatgctaaaatgagttgttcaatgttctcatagttatctctattcacccagtcaagttcattaataaatacactaatgttcaaattaacacaggacaaaatcagtttgctgcttacaaagccaacatccaagtcatccagattcattgtcctagaagtgttattctttttgcagtcacaaatgaactgggttaattgtttcagatcatgttgtgcattgtttggcaacaattcaagctcaccaaaccaaaaatatttcttgaactgagatgttgacataatcacaggcaccaacattgactcaaacaaaatctgtatcaagaaatttgtgcacacttcttctggttcaaggttgaatcctctctccagtggatgagactctctgctatgggacattgcaagctcattttgctttacaatatacaattcttctctgcgatgttttataatatgactaacaataccaagacattctgatgttatatcaattgccacacaaaggtctaagaactttatcctctgaacccatgatagcctcagcatattcaaatcagacaggaaaggggatatgtgttcatcaaatagtgtagggaagttcctcctgattgagtaaagtatgtggttgatgcccaccttgtcctcaagctcagaatgtgtgcttggttttattggccagaagtgattgggattgtttaggtgagtgactatcttgggtacttcagctttttgaaacacccagttacccaactcgcaagcattggttaacacaagagcaaaataatcccaaattaagggtctggagtactcacttacttcaccaagtgctgctttacaataaacacctttgcgctgattacaaaagtgacaatcacggtgtaagataatcttgcttgtaatatccctgatatacttaaatcctcctttcccgtctcttacacattttgagcccatacttttgcaaactcctatgaatcctgatgctatgctgctctgaaaagctgatttgttgatagcatcagccaaaatcttcttagcccctctgacatagttctttgataatttggactgtacggatttgacaagactgggtatttcttctcgctgcacagttcttgttgtgctcattaacttagtacgaagcaccaatctgagatcaccatgaacccttaaatttaaccacctaatattaagagcatcctcaatagcctcagtctcgacatcacaagtctctaataactgttttaagcagtcatccggtgattgctgaagagttgttacaatataactttcttccagggctccagactgtattttgtaaaatattttcctgcatgcctttctgattattgaaagtagcagatcatcaggaaatagtgtctcaattgatcgctgaagtctgtaccctctcgacccattaacccaatcgagtacatccatttcttccaggcacaaaaatggatcatttggaaacccactatagattatcatgctatttgttcgttttgcaatggcccctacaacctctattgacaccccgttagcaacacattggtccagtattgtgtcaattgtatctgcttgctgattgggtgctttagcctttatgttgtgtagagctgcagcaacaaactttgtaaggagggggacttcttgtgaccaaatgaagaatctcgatttgaactcacttgcaaaggtccccacaactgttttagggctcacaaacttgttgagtttgtctgatagaaagtagtgaaactccatacagtccaataccaattcaacattcaactcatctctgtccttaaatttgaaaccctcattcaaggataacatgatctcatcatcactcgaagtatatgagatgaaccgtgctccataacaaagctccaatgcgtaattgatgaactgctcagtgattagaccatataagtcagaggtgttgtgtaggatgccctgacccatatctaagactgaagagatgtgtgatggtaccttgcccttctcaaagtacccaaacataaattcctctgcaattgtgcacccccctttatccatcatacccaacccccttttcaagaaacctttcatgtatgcctcaacgacattgaagggcacttccaccatcttgtgaatgtgccatagcaatatgttgatgactgcagcattgggaacttctgacccatctttgagtttgaactcaagaccttttaataatgcggcaaagataaccggcgacatgtgtggcccccattttgaatggtccattgacaccgcaagaccactttgcctaacaactgacttcatgtctaataatgctctctcaaactctttctcgttgttcagacaagtatacctcatgttttgcataagggattcagagtaatcctcaatgagtctggttgtgagtttagtatttaaatcaccgacataaagctccctgttgccacccacctgttctttataagaaagaccaaatttcaatctccctacattggtggatacaccagacctctctgtgggagactcatctgaatagaaacagagatttcgtaaggatgagttggtaaaaaagctttgatccaatcttttagctatcgattcagaattgctctctcttgagcttatacgtgatgtctctctaatttgtagtgctgcatctgtgaacccaagtctgcttctacttttgtgatcatatcttccgactcgattatcataatcgcttgcaatgagaatgtatttaaagcactcaaaataatcagcttctttgtacgccttcaatgtgaggttctttattaaaaactccagaggacacggattcattagtctgtctgcaaagtacactgatctagcagtgacatcctcatagatcaagtttacaagatcctcatacacttctgctgaaaacaggctgtaatcaaaatcctttacatcatgaagtgaagtctctcttttgatgacaaccattgtcgatttgggccataatctctctagtggacatgaagtcttaaggttggttttgacattggtgtcaaccttagacaatacttttgcaactctggtctcaatttctttaagacagtcaccctgatcttctgatagtaactcttcaactccatcaggctctattgactccttttttatttggatcaatgatgacaacctcttcagaatcttgaaatttacctcctttggatccaacttgtatttacccttagttttgaaatgttcaatcatttccacaacaacagcagacacaatggaagagtaatcatattcagtgatgacctcaccaacttcattgagttttggaaccaccacacttttgttgctggacatatccaaggctgtacttgtgaaggagggagtcatagggtcacaaggaagcaggggtttcacttccaatgagctactgttaaatagtgatagacaaacactaagtacatccttattcaaccccggccttccctcacatttggattccagctttttaccaagtagtctctctatatcatgcaccatcttctcttcttcctcagtaggaagttccatactattagaagggttgaccaagactgaatcaaactttaactttggttccaagaacttctcaaaacatttgatttgatcagttaatctatcaggggtttctttggttataaaatggcataaataggagacattcaaaacaaacttaaagatcttagccatatcttcctctctggagttgctgagtaccagaagtatcaaatcatcaataagcattgctgtctgccattctgaaggtgttagcataacgactttcaatttctcaaacaattctttaaaatgaacttcatttacaaaggccataatgtaatatctaaagccttgcaagtaaacttgaatacgcttggaaggggtgcacagtatgcagagaataagtcgtctgagtaaatcagaaacagaatccaagaggggttgggacataaagtccaaccaggataacatctccacacaagtcctttgaatcacatctgcactaaagatcggtaagaaaaatctcttgggatcacagtaaaaagacgcttttgtttcatacaaacccccacttttggatctataagcaacagcataacacctggacctctcccctgtcttctggtacagtagtgtgagagaacctccttctccaaatcgctggaagaaaacttcgtcacagtaaaccttcccataaaactcatcagcattgttcaccttcatcttaggaactgctgctgtcttcatgctattaatgagtgacaaactcaaacttgacaatgttttcagcaattcctcaaactcactttcgcccatgatggtataatcaggctgccctcttcctggcctacccccacacatacactgtgactttgtcttgtattgaagacagggtttagcaccccattcatctaacactgatgttttcagattgaagtaatattcaacatcaggttcccgtagaagagggagaatgtcatcaaggggaagttcaccacagaccgagctcagtctcttcttagccttctctaaccagttggggtttttaatgaattttttagtgatttgttccatcaggaagtcgacattaatcaacctgtcatttacagacggtaacccttgcattaggagcacctctctgaacacagcacctggagaagacttgtccaagtcacacaaaatgttgtacatgataaggtccagaaccaacatggtgttcctccttgtgttaaaaaccttttgagacttaattttgttgcatattgaaagtactctaaaatattctctgctttcagttgatgaatgcttgacctcagattgcctgagttggcctattatgcccaaaatgtgtactgagcaaaactcacataatctgatttctgatttaggtacatctttgacagaacattggataaattcatggttctgaagtctagaaatcatatcttccctatctgtagcctgcagtttcctatcgagttgaccagcaagttgcaacattttaaattgctgaaagatttccatgatttttgttctacattgatctgttgtcagtttattattaatgccagacattaatgccttttccaacctcactttgtaaggaagtcccctttcctttacagcaagtagtgactccagaccgagactctgattttctaaggatgagagggaacttataaggcgttcgtactccaactcctcaacttcttcaccagatgtccttaatccatccatgagttttaaaagcaaccaccgaagtctctctaccacccaatcaggaacaaattctacataataactggatctaccgtcaataacaggtactaaggttatgttctgtctcttgagatcagaactaagctgcaacagcttcaaaaagtcctggttgtatttcttctcaaatgcttcttgactggtcctcacaaacacttccaaaagaatgaggacatctccaaccatacagtaaccatctggtgtaacatccggcaatgtaggacatgttactctcaactccctaaggatagcattgacagtcatctttgtgttgtgtttgcaggagtgtttcttgcatgaatccacttccactagcatggacaaaagcttcaggccctctatcgtgatggccctatctttgacttgtgcaagaacgttgtttttctgttcagatagctcttcccattcgggaacccattttctgactatgtctttaagttcgaaaacgtattcctccatgatcaagaaatgcctaggatcctcggtgcg

SEQ ID NO: 22 - RNA, 3421 nt, Pichinde virus
  source 1..3421: mol_type = genomic RNA
cgcaccggggatcctaggcataccttggacgcgcatattacttgatcaaagatgggacaagttgtgactttgatccagtctatacccgaagtcctgcaggaggtcttcaatgtcgccttaatcattgtctcaaccctatgcatcatcaaaggatttgtcaatctgatgagatgtggcctattccaactcatcaccttcctcattttggctggcagaagttgtgatggcatgatgattgataggaggcacaatctcacccacgttgagttcaacctcacaagaatgtttgacaacttgccacaatcatgtagcaagaacaacacacatcattactacaaaggaccatctaacacaacatggggaattgaactcactttgacaaacacatccattgcaaatgaaactactggaaacttttccaacatcagaagccttgcatatggtaacattagtaattgtgataagacagaagaagcaggtcacacattaaaatggttgcttaatgagttacacttcaatgtgctccatgtcactcgtcatgtaggtgccagatgcaaaacagttgagggtgctggggtgttgatccagtacaacttgacagttggggatagaggaggtgaggttggcagacatcttattgcgtcgcttgctcaaatcattggggacccaaaaattgcgtgggttggaaaatgtttcaataactgtagtggagggtcttgcagactaacaaactgtgaaggtgggacacattacaatttcctgatcatacagaacaccacatgggaaaatcactgtacatatactccaatggcaacaataaggatggctctccaaaaaactgcttatagttctgtgagcaggaaactccttggctttttcacttgggacttgagtgactctactgggcaacatgtcccaggtggttactgtttggagcaatgggctattgtttgggctggaataaaatgttttgataacactgtgatggcaaaatgcaacaaagatcacaatgaagaattttgcgatacgatgaggttatttgatttcaatcagaatgctatcaaaaccttacaacttaatgttgagaattcgttgaatctctttaaaaagactatcaacggacttatttctgactcacttgtgattagaaacagtctcaaacagcttgccaaaatcccttattgcaactatacaaaattttggtacatcaatgataccatcacaggaagacattctttaccgcagtgttggttagttcacaatggctcgtacctcaatgaaacgcattttaagaatgattggttgtgggagagccagaatctgtacaatgaaatgctgataaaagaatatgaagaaagacaaggtaagactccactagcattgacagacatttgcttctggtctttggtgttttacaccatcacagtgtttctccacttagttggaatacccactcataggcacatcattggtgatggctgtccgaagccacataggattactaggaactctctttgcagctgtgggtattataaaatcccaaagaaaccctacaaatgggtgagactgggtaaataagccctagcctcgacatgggcctcgacgtcactccccaataggggagtgacgtcgaggcctctgaggacttgagctcagaggttgatcagatctgtgttgttcctgtacagcgtgtcaataggcaagcatctcatcggcttctggtccctaacccagcctgtcactgttgcatcaaacatgatggtatcaagcaatgcacagtgaggattcgcagtggtttgtgcagcccccttcttcttcttctttatgaccaaacctttatgtttggtgcagagtagattgtatctctcccagatctcatcctcaaaggtgcgtgcttgctcggcactgagtttcacgtcaagcacttttaagtctcttctcccatgcatttcgaacaaactgattatatcatctgaaccttgagcagtgaaaaccatgttttgaggtaaatgtctgatgattgaggaaatcaggcctggttgggcatcagccaagtcctttaaaagaagaccatgtgagtacttgctttgctctttgaaggacttctcatcgtggggaaatctgtaacaatgtatgtagttgcccgtgtcaggctggtagatggccatttccaccggatcatttggtgttccttcaatgtcaatccatgtggtagcttttgaatcaagcatctgaattgaggacacaacagtgtcttctttctccttagggatttgtttaaggtccggtgatcctccgtttcttactggtggctggatagcactcggcttcgaatctaaatctacagtggtgttatcccaagccctcccttgaacttgagaccttgagccaatgtaaggccaaccatcccctgaaagacaaatcttgtatagtaaattttcataaggatttctctgtccgggtgtagtgctcacaaacataccttcacgattctttatttgcaatagactctttatgagagtactaaacatagaaggcttcacctggatggtctcaagcatattgccaccatcaatcatgcaagcagctgctttgactgctgcagacaaactgagattgtaccctgagatgtttatggctgatggctcattactaatgatttttagggcactgtgttgctgtgtgagtttctctagatctgtcatgttcgggaacttgacagtgtagagcaaaccaagtgcactcagcgcttggacaacatcattaagttgttcacccccttgctcagtcatacaagcgatggttaaggctggcattgatccaaattgattgatcaacaatgtattatccttgatgtcccagatcttcacaaccccatctctgttgcctgtgggtctagcattagcgaaccccattgagcgaaggatttcggctctttgttccaactgagtgtttgtgagattgcccccataaacaccaggctgagacaaactctcagttctagtgactttctttcttaacttgtccaaatcagatgcaagctccattagctcctctttggctaagcctcccaccttaagcacattgtccctctggattgatctcatattcatcagagcatcaacctctttgttcatgtctcttaacttggtcagatcagaatcagtccttttatctttgcgcatcattctttgaacttgagcaactttgtgaaagtcaagagcagataacagtgctcttgtgtccgacaacacatcagccttcacaggatgggtccagttggatagacccctcctaagggactgtacccagcggaatgatgggatgttgtcagacattttggggttgtttgcacttcctccgagtcagtgaagaagtgaacgtacagcgtgatctagaatcgcctaggatccactgtgcg

(19) United States
(12) Patent Application Publication
(10) Pub. No.: US 2026/0218232 A1
(43) Pub. Date: Jul. 30, 2026

(54) MULTIVALENT VACCINES AGAINST TUBERCULOSIS

(71) Applicant: REGENTS OF THE UNIVERSITY OF MINNESOTA, Minneapolis, MN (US)

(72) Inventors: Yuying Liang, Woodbury, MN (US); Hinh Ly, Woodbury, MN (US); Anna D. Tischler, Golden Valley, MN (US)

(21) Appl. No.: 19/146,516

(22) PCT Filed: Jan. 17, 2024
(86) PCT No.: PCT/US2024/011727

§ 371 Date: Jul. 8, 2025

Related U.S. Application Data
(60) Provisional application No. 63/439,308, filed on Jan. 17, 2023.

(51) Int. Cl. C12N 15/86; A61K 39/00; A61K 39/12; A61P 37/04; C07K 14/35; C12N 9/12

(52) U.S. Cl. CPC C12N 15/86; A61K 39/12; A61P 37/04; C07K 14/35; C12N 9/127; C12Y 207/07048; A61K 2039/5258; A61K 2039/575; C12N 2760/10043; C12N 2760/10052

(57) ABSTRACT

Provided herein are genetically engineered Pichinde viruses that include three ambisense genomic segments. The first genomic segment includes a coding region encoding a Z protein and a coding region encoding a L RdRp protein. The second genomic segment includes a coding region encoding a nucleoprotein (NP) and the third genomic segment includes a coding region encoding a glycoprotein. Each of the second and third genomic segments optionally include an additional coding region that may encode one or more Mycobacterium tuberculosis proteins. Also provided herein is a reverse genetics system for making a genetically engineered Pichinde virus, and a collection of vectors that can be used to produce a genetically engineered Pichinde virus. Further provided are methods for using a reverse genetics system, and methods for producing an immune response in a subject.

### CROSS-REFERENCE TO RELATED APPLICATIONS

This application claims the benefit of U.S. Provisional Application Ser. No. 63/439,308, filed Jan. 17, 2023, which is incorporated by reference herein in its entirety.

### SEQUENCE LISTING

This application contains a Sequence Listing electronically submitted via Patent Center to the United States Patent and Trademark Office as an XML file entitled “0110-000711WO01-SEQ.xml” having a size of 39,251 bytes and created on Jan. 17, 2024.

The information contained in the Sequence Listing is incorporated by reference herein.

### BACKGROUND

Tuberculosis (TB) is a predominantly pulmonary disease caused by Mycobacterium tuberculosis (Mtb), with more than 10 million new cases of active disease and 1.6 million deaths attributed to the infection in 2021 (World Health Organization Global tuberculosis report 2022, www.who.int/publications-detail-redirect/9789240061729). Mtb is also estimated to cause asymptomatic latent infections in one quarter of the world's population (Houben and Dodd, PLoS Med (2016) 13:e1002152. doi: 10.1371/journal.pmed.1002152), each of whom has a 5-10% lifetime risk of developing active TB disease (World Health Organization Global Tuberculosis Report 2021, www.who.int/teams/global-tuberculosis-programme/tb-reports/global-tuberculosis-report-2021). The only licensed TB vaccine, Bacille Calmette-Guerin (BCG), is effective at preventing severe childhood meningeal and miliary TB, but does not prevent adult pulmonary TB (Andersen and Kaufmann, Cold Spring Harb Perspect Med (2014) 4: doi: 10.1101/cshperspect.a018523; Mangtani et al., Clin Infect Dis (2014) 58:470-480. doi: 10.1093/cid/cit790). With multidrug-resistant (MDR) and extensively drug-resistant (XDR) Mtb strains becoming more common, development of new and effective TB vaccines is a top global health priority (World Health Organization Global tuberculosis report 2022, www.who.int/publications-detail-redirect/9789240061729). This need was highlighted in 2020 during the COVID-19 pandemic, which caused disruptions in disease detection and reporting that led to increased TB deaths (World Health Organization Global Tuberculosis Report 2021, www.who.int/teams/global-tuberculosis-programme/tb-reports/global-tuberculosis-report-2021). TB vaccine development has accelerated in recent years with several whole cell-derived and subunit vaccines in current or recently completed clinical trials. A recent phase 2b clinical trial of the recombinant fusion protein vaccine candidate (M72/AS01E) in latently infected adults elicited immune responses and provided 54.0% protection from active TB disease over a 3-year study period (Tait et al., N Engl J Med (2019) 381:2429-2439. doi: 10.1056/NEJMoa1909953).

### SUMMARY OF THE APPLICATION

Tuberculosis (TB) caused by Mycobacterium tuberculosis (Mtb) is one of the leading infectious disease killers worldwide despite the availability of antibiotics and the Bacille Calmette-Guérin (BCG) vaccine. New TB vaccines that can prevent adult pulmonary TB are urgently needed to combat this worldwide epidemic. A viral vaccine vector based on recombinant Pichinde virus (PICV) and demonstrated its safety, versatility, and strong immune efficacy for vaccine development has been reported (WO 2016/048949). In the present disclosure several PICV-vectored TB vaccines have been generated and shown to elicit strong systemic and mucosal memory T cell responses. The TB vaccines provide protection against virulent Mtb challenge in mice.

Terms used herein will be understood to take on their ordinary meaning in the relevant art unless specified otherwise. Several terms used herein and their meanings are set forth below.

As used herein, “genetically modified” and “genetically engineered” refers to a Pichinde virus which has been modified and is not found in any natural setting. For example, a genetically modified Pichinde virus is one into which has been introduced an exogenous polynucleotide, such as a restriction endonuclease site. Another example of a genetically modified Pichinde virus is one which has been modified to include three genomic segments.

A “coding region” is a nucleotide sequence that encodes an RNA molecule. The boundaries of a coding region are generally determined by a transcription initiation site at its 5′ end and a transcription terminator at its 3′ end. A coding region typically includes at least one nucleotide sequence that encodes a protein. A nucleotide sequence encoding a protein, also referred to as an open reading frame (ORF) has boundaries that are generally determined by a translation start codon at its 5′ end and a translation stop codon at its 3′ end. A coding region can encode an RNA molecule that includes one or more open reading frames. An RNA molecule that includes one open reading frame is referred to as a “monocistronic message.” An RNA molecule that includes two or more open reading frames is referred to as a “polycistronic message.”

As used herein, the term “protein” refers broadly to a polymer of two or more amino acids joined together by peptide bonds. The term “protein” also includes molecules which contain more than one protein joined by disulfide bonds, ionic bonds, or hydrophobic interactions, or complexes of proteins that are joined together, covalently or noncovalently, as multimers (e.g., dimers, tetramers). Thus, the terms peptide, oligopeptide, and polypeptide are all included within the definition of protein and these terms are used interchangeably. It should be understood that these terms do not connote a specific length of a polymer of amino acids, nor are they intended to imply or distinguish whether the protein is produced using recombinant techniques, chemical or enzymatic synthesis, or is naturally occurring.

As used herein, “ex vivo” refers to a cell that has been removed from the body of an animal. Ex vivo cells include, for instance, primary cells (e.g., cells that have recently been removed from a subject and are capable of limited growth in tissue culture medium), and cultured cells (e.g., cells that are capable of long-term culture in tissue culture medium). “In vivo” refers to cells that are within the body of a subject.

While the polynucleotide sequences described herein are listed as DNA or RNA sequences, it is understood that the complements, reverse sequences, and reverse complements of the DNA and RNA sequences can be easily determined by the skilled person. It is also understood that the sequences disclosed herein as DNA sequences can be converted from a DNA sequence to an RNA sequence by replacing each thymidine nucleotide with a uridine nucleotide. Likewise, the sequences disclosed herein as RNA sequences can be converted from a RNA sequence to an DNA sequence by replacing each uridine nucleotide with a thymidine nucleotide.

Unless otherwise specified, “a,” “an,” “the,” and “at least one” are used interchangeably and mean one or more than one.

As used in this specification and the appended claims, the term “or” is generally employed in its sense including “and/or” unless the content clearly dictates otherwise. The term “and/or” means one or all of the listed elements or a combination of any two or more of the listed elements. The use of “and/or” in some instances does not imply that the use of “or” in other instances may not mean “and/or.”

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

As used herein, “have,” “has,” “having,” “include,” “includes,” “including,” “comprise,” “comprises,” “comprising” or the like are used in their open ended inclusive sense, and generally mean “include, but not limited to,” “includes, but not limited to,” or “including, but not limited to.”

It is understood that wherever embodiments are described herein with the language “have,” “has,” “having,” “include,” “includes,” “including,” “comprise,” “comprises,” “comprising” and the like, otherwise analogous embodiments described in terms of “consisting of” and/or “consisting essentially of” are also provided. The term “consisting of” means including, and limited to, whatever follows the phrase “consisting of.” That is, “consisting of” indicates that the listed elements are required or mandatory, and that no other elements may be present. The term “consisting essentially of” indicates that any elements listed after the phrase are included, and that other elements than those listed may be included provided that those elements do not interfere with or contribute to the activity or action specified in the disclosure for the listed elements.

Conditions that are “suitable” for an event to occur, such as generating full-length genomic RNA molecules of a genomic segment, or “suitable” conditions are conditions that do not prevent such events from occurring. Thus, these conditions permit, enhance, facilitate, and/or are conducive to the event.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

In the description herein particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The following detailed description of illustrative embodiments of the present disclosure may be best understood when read in conjunction with the following drawings.

FIG. 1A-B shows generation of rP18tri-based viral vector vaccines encoding Mtb antigens. (FIG. 1A) Genomic organization of the viral vectored TB vaccines (TBvac-1, TBvac-2 and TBvac-10), with the inserted Mtb antigens shown below. The P2A linker sequence was used to express two antigens from one ORF. (FIG. 1B) Detection of Mtb Ag85B antigen expression by Western blotting. Vero cell lysates collected 24 hours after mock infection or infection with vector, TBvac-1, or TBvac-2 were analyzed by Western blotting with anti-Ag85B and anti-GAPDH antibodies.

FIG. 2A-C show evaluation of TBvac2-induced T cell responses by ELISPOT assay. (FIG. 2A) BL6 mice (N=3) were primed with 1×105 PFU of rP18tri viral vector (V) alone or TBvac-2 through the IM route, and 21 days later boosted with the same virus at the same dose through the IN route. At 7 d post-boost (dpb), splenocytes were isolated and incubated with medium alone or peptide pools of Ag85B or ESAT-6/EsxA, followed by detection of mouse IFNgamma by ELISpot. (FIG. 2B) Representative ELISpot wells. (FIG. 2C) The number of IFNγ-secreting cells per million splenocytes in the vector and TBvac-2-immunized mice after Ag85B or ESAT-6/EsxA peptide stimulation was normalized with medium-alone control and shown as the average with standard deviation. Statistical analysis was conducted with unpaired t-test. **, p<0.01; *, p<0.05; ns, not significant.

FIG. 3A-F show rP18tri-based TB vaccines induced strong antigen-specific CD8 T cells through both IM and IN routes. (FIG. 3A) BL6 mice (N=4-5) were immunized with PBS (M), rP18tri vector alone (V), or TBvac-2 through either IM or IN route. Lymphocytes from peripheral blood (PBMC), spleen, and lung were incubated with PE-labeled MHC-I EsxH tetramer, along with antibodies against surface markers (CD3, CD8, and CD44), and analyzed by flow cytometry. Representative flow cytometry plots of the tetramer-positive CD44+CD8 T cells in peripheral blood (PBMC), spleens, and lungs from vector and TBvac-2-immunized mice are shown (FIG. 3B). The percentage of tetramer-positive CD44+CD8T cells in PBMCs (FIG. 3C) and spleens (FIG. 3D) is shown for each group (M, V, and TBvac-2) at 7 dpp through IM or IN route. The percentage of tetramer-positive CD44+CD8T cells in lungs (FIG. 3E) is shown for each group (M, V, and TBvac-2) at 7 days post-boost (dpb) through IM-IM or IN-IN route. A similar study was conducted to evaluate the TBvac-10-induced CD8T cells. BL6 mice (N=5) were immunized with rP18tri vector (V) or TBvac-10 through IM-IN prime-boost strategy. Representative flow cytometry plots (FIG. 3F) and the percentage (FIG. 3G) of tetramer-positive CD44+CD8T cells at 7 dpb in PBMCs, spleens, and lungs from vector and TBvac-10-immunized mice are shown. Statistical analysis was conducted with unpaired t-test. ***, p<0.001; **, p<0.01; *, p<0.05; ns, not significant.

FIG. 4A-E show rP18tri-based TB vaccines induced strong antigen-specific CD4 T cells. (FIG. 4A) BL6 mice (N=5) were immunized with rP18tri vector (V), TBvac-2, and TBvac-10 through IM-IN or IN-IN prime-boost strategy. At 7 dpb, lymphocytes isolated from spleens or lungs were incubated with a PE-labeled control or Ag85B MHC-II tetramer, together with antibodies against cell surface markers (CD3, CD4, CD8, CD44, CD69). (FIG. 4B) Gating strategy used to analyze the MHC-I tetramer-positive CD44+CD4+ T cells and lung CD69+CD44+CD4+ T cells. (FIG. 4C) Representative flow cytometry plots of control- and Ag85b-tetramer-positive CD44+CD4+ T cells were shown for splenocytes of vector and TBvac-2 immunization, and for lung lymphocytes in vector and TBvac-10 immunization. After normalization with the control MHC-11 tetramer, the Ag85B tetramer-positive spleen CD44+CD4+ T cells (FIG. 4D), lung CD44+CD4+ T cells (FIG. 4E), and lung CD69+CD44+CD4+ T cells (FIG. 4F) was shown for vector (V), TBvac-2, and TBvac-10 immunization. Statistical analysis was conducted with unpaired t-test. ***, p<0.001; **, p<0.01; *, p<0.05; ns, not significant.

FIG. 5A-C show rP18tri-based TB vaccines induced multifunctional CD4 T cells. (FIG. 5A) BL6 mice (N=5) were immunized with rP18tri vector (V), TBvac-2, and TBvac-10 through IM-IN or IN-IN prime-boost strategy. At 7 dpb, PBMCs were collected and incubated in medium only or with pooled Ag85B/ESAT6 antigens, and stained for cell surface markers and cytokines. Representative flow cytometry plots of memory CD4 T cells (CD44+CD4+CD3+) positive for dual cytokine expression (IFNgamma/IL-2, IFNgamma/TNFalpha, and IL-2/TNFalpha) after either medium or peptide stimulation were shown for each immunization group (FIG. 5B). After normalization with the medium control, the average percentages of dual- and triple-positive (IFNgamma/IL-2/TNFalpha) memory CD4 T cells after peptide stimulation were shown for each group (FIG. 5C).

FIG. 6A-B show TBvac-1 and TBvac-2 reduced bacterial load in mouse lungs in a virulent Mtb infection model. (FIG. 6A) Illustration of the experimental procedure to evaluate the virus-induced protective efficacy in mice. Mice (n=4-5) were immunized (IM) through prime-boost strategy with vector or TBvac-1 or TBvac-2, and, 28 days later, challenged with virulent Mtb. (FIG. 6B) Tissues (lungs and spleens) were collected at 4 and 12 weeks after challenge to determine viable Mtb CFU by plating of tissue homogenates. Statistical analysis was conducted with unpaired t-test. **, p<0.01; *, p<0.05; ns, not significant. Triangles, vector; squares, TBvac-1; circles, TBvac-2.

FIG. 7 shows certain amino acid and nucleic acid sequences discussed in the present disclosure.

The schematic drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar to other numbered components.

### DETAILED DESCRIPTION

Provided herein is a reverse genetics system for producing genetically modified Pichinde virus that expresses proteins of Mycobacterium tuberculosis (Mtb). The genetically modified Pichinde virus-based reverse genetics system described herein has multiple advantages over other arenavirus systems for expression of Mtb proteins and use in immunizing subjects. Pichinde virus is not known to cause disease in humans, and there is evidence that Pichinde virus can cause asymptomatic human infections in a laboratory setting. For instance, 46% of laboratory personnel working with the virus are serum positive but do not show a distinct illness (Buchmeier et al., 2007, Arenaviridae: the viruses and their replication. In: Knipe and Howley (eds), Fields Virology. 5th ed. Philadelphia, PA: Lippincott Williams & Wilkins. pp. 1791-1827). The modified Pichinde virus described herein is further attenuated in comparison to the parental virus used in the human-infection study reported by Buchmeier et al. The modified Pichinde virus is genetically stable through serial passages in cell cultures. General human populations are not known to have prior exposure to Pichinde virus, which makes it an ideal vector for vaccine development due to the lack of pre-existing immunity against this Pichinde virus vector.

The reverse genetics system for this modified Pichinde virus includes three genomic segments. The first genomic segment includes two coding regions, one that encodes a Z protein and a second that encodes a RNA-dependent RNA polymerase (L RdRp). The second genomic segment includes a coding region that encodes a nucleoprotein (NP), and one or more additional coding regions that encode one or more Mtb proteins. The third genomic segment includes a coding region that encodes a glycoprotein, and one or more additional coding regions that encode one or more Mtb proteins.

The Z protein, L RdRp, NP protein, and glycoprotein are those encoded by a Pichinde virus. The Z protein is a small RING-domain containing matrix protein that mediates virus budding and also regulates viral RNA synthesis. One example of a Z protein from a Pichinde virus is the sequence available at Genbank accession number ABU39910.1 (SEQ ID NO:1). The L RdRp protein is a RNA-dependent RNA polymerase that is required for viral DNA synthesis. One example of a L RdRp protein from a Pichinde virus is the sequence available at Genbank accession number ABU39911.1 (SEQ ID NO:2). The NP protein encapsidates viral genomic RNAs, is required for viral RNA synthesis, and also suppresses host innate immune responses. One example of a NP protein from a Pichinde virus is the sequence available at Genbank accession number ABU39909.1 (SEQ ID NO:3). The glycoprotein is post-translationally processed into a stable signal peptide (SSP), the receptor-binding G1 protein, and the transmembrane G2 protein. One example of a glycoprotein from a Pichinde virus is the sequence available at Genbank accession number ABU39908.1 (SEQ ID NO:4).

Other examples of Z proteins, L RdRp proteins, NP proteins, and glycoprotein include proteins having structural similarity with a protein that is encoded by a Pichinde virus, for instance, SEQ ID NO:1, 2, 3, and/or 4. Structural similarity of two proteins can be determined by aligning the residues of the two proteins (for example, a candidate protein and a reference protein described herein) to optimize the number of identical amino acids along the lengths of their sequences; gaps in either or both sequences are permitted in making the alignment in order to optimize the number of identical amino acids, although the amino acids in each sequence must nonetheless remain in their proper order. A reference protein may be a protein described herein, such as SEQ ID NO:1, 2, 3, or 4. A candidate protein is the protein being compared to the reference protein. A candidate protein may be isolated, for example, from a cell of an animal, such as a mouse, or can be produced using recombinant techniques, or chemically or enzymatically synthesized. A candidate protein may be inferred from a nucleotide sequence present in the genome of a Pichinde virus.

Unless modified as otherwise described herein, a pair-wise comparison analysis of amino acid sequences can be carried out using the Blastp program of the blastp suite-2sequences search algorithm, as described by Tatiana et al., (FEMS Microbiol Lett, 174, 247-250 (1999)), and available on the National Center for Biotechnology Information (NCBI) website. The default values for all blastp suite-2sequences search parameters may be used, including general parameters: expect threshold=10, word size=3, short queries=on; scoring parameters: matrix=BLOSUM62, gap costs=existence:11 extension:1, compositional adjustments=conditional compositional score matrix adjustment. Alternatively, proteins may be compared using the BESTFIT algorithm in the GCG package (version 10.2, Madison WI).

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions. A conservative substitution for an amino acid in a protein described herein may be selected from other members of the class to which the amino acid belongs. For example, it is known in the art of protein biochemistry that an amino acid belonging to a grouping of amino acids having a particular size or characteristic (such as charge, hydrophobicity and hydrophilicity) can be substituted for another amino acid without altering the activity of a protein, particularly in regions of the protein that are not directly associated with biological activity. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Conservative substitutions include, for example, Lys for Arg and vice versa to maintain a positive charge; Glu for Asp and vice versa to maintain a negative charge; Ser for Thr so that a free —OH is maintained, and Gln for Asn to maintain a free —NH2.

The skilled person will recognize that the Z protein depicted at SEQ ID NO:1 can be compared to Z proteins from other arenaviruses, including Lassa virus (073557.4), LCMV Armstrong (AAX49343.1), and Junin virus (NP_899216.1) using readily available algorithms such as ClustalW to identify conserved regions of Z proteins. ClustalW is a multiple sequence alignment program for nucleic acids or proteins that produces biologically meaningful multiple sequence alignments of different sequences (Larkin et al., 2007, ClustalW and ClustalX version 2, Bioinformatics, 23(21):2947-2948). Using this information, the skilled person can readily predict with a reasonable expectation that certain conservative substitutions to an Z protein such as SEQ ID NO:1 will not decrease activity of the protein.

The skilled person will recognize that the L RdRp protein depicted at SEQ ID NO:2 can be compared to L RdRp proteins from other arenaviruses, including Lassa virus (AAT49002.1), LCMV Armstrong (AAX49344.1), and Junin virus (NP_899217.1) using readily available algorithms such as ClustalW to identify conserved regions of L RdRp proteins. Using this information the skilled person can readily predict with a reasonable expectation that certain conservative substitutions to an L RdRp protein such as SEQ ID NO:2 will not decrease activity of the protein.

The skilled person will recognize that the NP protein depicted at SEQ ID NO:3 can be compared to NP proteins from other arenaviruses, including Lassa virus (P13699.1), LCMV Armstrong (AAX49342.1), and Junin virus (NP_899219.1) using readily available algorithms such as ClustalW to identify conserved regions of NP proteins. Using this information the skilled person can readily predict with a reasonable expectation that certain conservative substitutions to a NP protein such as SEQ ID NO:3 will not decrease activity of the protein.

The skilled person will recognize that the glycoprotein depicted at SEQ ID NO:4 can be compared to glycoproteins from other arenaviruses, including Lassa virus (P08669), LCMV Armstrong (AAX49341.1), and Junin virus (NP_899218.1) using readily available algorithms such as ClustalW to identify conserved regions of glycoproteins. Using this information the skilled person can readily predict with a reasonable expectation that certain conservative substitutions to a glycoprotein such as SEQ ID NO:4 will not decrease activity of the protein.

Thus, as used herein, a Pichinde virus Z protein, L RdRp protein, an NP protein, or a glycoprotein includes those with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence similarity to a reference amino acid sequence. Alternatively, as used herein, a Pichinde virus Z protein, L RdRp protein, an NP protein, or a glycoprotein includes those with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to a reference amino acid sequence. Unless noted otherwise, “Z protein,” “L RdRp protein,” “NP protein,” and “glycoprotein” refer to a protein having at least 80% amino acid identity to SEQ ID NO:1, SEQ ID NO:2, SEQ ID NO:3, and SEQ ID NO:4, respectively.

A Z protein, L RdRp protein, an NP protein, or a glycoprotein having structural similarity the amino acid sequence of SEQ ID NO:1, 2, 3, or 4, respectively, has biological activity. As used herein, “biological activity” refers to the activity of Z protein, L RdRp protein, an NP protein, or a glycoprotein in producing an infectious virus particle. The biological role each of these proteins play in the biogenesis of an infectious virus particle is known, as are assays for measuring biological activity of each protein.

In one embodiment, the NP protein may include one or more mutations. A mutation in the NP protein may result in a NP protein that continues to function in the production of infectious viral particles, but has a decreased ability to suppress the production of certain cytokines by a cell infected with a Pichinde virus. A Pichinde virus that has decreased ability to suppress cytokine production is expected to be useful in enhancing an immunological response to a protein encoded by the virus. Examples of mutations include the aspartic acid at residue 380, the glutamic acid at residue 382, the aspartic acid at residue 457, the aspartic acid at residue 525, and the histidine at residue 520. A person of ordinary skill in the art recognizes that the precise location of these mutations can vary between different NP proteins depending upon the presence of small insertions or deletions in the NP protein, thus the precise location of a mutation is approximate, and can vary by 1, 2, 3, 4, 5, or more amino acids.

In one embodiment, the mutation in the NP protein may be the replacement of the aspartic acid, glutamic acid, or histidine at residues 380, 382, 457, 525, and/or 520 with any other amino acid. In one embodiment, the mutation may be the conservative substitution of the aspartic acid, glutamic acid, or histidine at residues 380, 382, 457, 525, and/or 520. In one embodiment, the mutation may be the replacement of the aspartic acid, glutamic acid, or histidine at residues 380, 382, 457, 525, and/or 520 with a glycine or an alanine. In one embodiment, the NP protein may include a mutation at one, two, three, or four of the residues 380, 382, 457, 525, or 520, and in one embodiment the NP protein may include a mutation at all five residues.

In one embodiment, the glycoprotein may include one or more mutations. A mutation in the glycoprotein may result in a glycoprotein that impairs virus spreading in vivo. Examples of mutations include the asparagine at residue 20, and/or the asparagine at residue 404. A person of ordinary skill in the art recognizes that the precise location of these mutations can vary between different glycoproteins depending upon the presence of small insertions or deletions in the glycoprotein, thus the precise location of a mutation is approximate, and can vary by 1, 2, 3, 4, or 5 amino acids.

In one embodiment, the mutation in the glycoprotein may be the replacement of the asparagine residue 20 and/or 404 with any other amino acid. In one embodiment, the mutation may be the conservative substitution of the asparagine residue 20 and/or 404. In one embodiment, the mutation may be the replacement of the asparagine residue 20 and/or 404 with a glycine or an alanine.

Proteins as described herein also may be identified in terms the polynucleotide that encodes the protein. Thus, this disclosure provides polynucleotides that encode a protein as described herein or hybridize, under standard hybridization conditions, to a polynucleotide that encodes a protein as described herein, and the complements of such polynucleotide sequences. As used herein, the term “polynucleotide” refers to a polymeric form of nucleotides of any length, either ribonucleotides or deoxynucleotides, and includes both double- and single-stranded DNA and RNA. A polynucleotide may include nucleotide sequences having different functions, including for instance coding sequences, and non-coding sequences such as regulatory sequences. A polynucleotide can be obtained directly from a natural source, or can be prepared with the aid of recombinant, enzymatic, or chemical techniques. A polynucleotide can be linear or circular in topology. A polynucleotide can be, for example, a portion of a vector, such as an expression or cloning vector, or a fragment. An example of a polynucleotide is a genomic segment.

An example of a polynucleotide encoding a Z protein is the nucleotides 85-372 of the sequence available at Genbank accession number EF529747.1 (SEQ ID NO:21), an example of a polynucleotide encoding an L RdRp protein is the complement of nucleotides 443-7027 of the sequence available at Genbank accession number EF529747.1 (SEQ ID NO:21), an example of a polynucleotide encoding an NP protein is the complement of nucleotides 1653-3338 of the sequence available at Genbank accession number EF529746.1 (SEQ ID NO:22), and an example of a polynucleotide encoding a glycoprotein protein is the nucleotides 52-1578 of the sequence available at Genbank accession number EF529746.1 (SEQ ID NO:22). It should be understood that a polynucleotide encoding a Z protein, an L RdRp protein, an NP protein, or a glycoprotein represented by SEQ ID NO:1, 2, 3, or 4, respectively, is not limited to the nucleotide sequence disclosed at SEQ ID NO:21 or 22, but also includes the class of polynucleotides encoding such proteins as a result of the degeneracy of the genetic code. For example, the naturally occurring nucleotide sequence SEQ ID NO:21 is but one member of the class of nucleotide sequences encoding a protein having the amino acid sequence SEQ ID NO:1 and a protein having the amino acid sequence SEQ ID NO:2. The class of nucleotide sequences encoding a selected protein sequence is large but finite, and the nucleotide sequence of each member of the class can be readily determined by one skilled in the art by reference to the standard genetic code, wherein different nucleotide triplets (codons) are known to encode the same amino acid.

| SEQ ID NO: 21: | |
|---|---|
| 1 | CGCACCGGGG ATCCTAGGCA TCTTTGGGTC ACGCTTCAAA TTTGTCCAAT TTGAACCCAG |
| 61 | CTCAAGTCCT GGTCAAAACT TGGGATGGGA CTCAGATATA GCAAAGAGGT CAGGAAGAGA |
| 121 | CATGGCGACG AAGATGTGGT GGGAAGGGTC CCCATGACCC TCAATCTACC ACAGGGCCTG |
| 181 | TATGGCAGGT TCAACTGCAA ATCTTGCTGG TTCGTCAACA AAGGTCTCAT CAGGTGCAAA |
| 241 | GACCACTATC TGTGTCTTGG GTGCTTAACC AAAATGCACT CCAGAGGCAA TCTCTGCGAG |
| 301 | ATATGCGGCC ACTCACTGCC AACCAAGATG GAGTTCCTAG AAAGCCCCTC TGCACCACCC |
| 361 | TACGAGCCAT AAACCAGGGC CCCTGGGCGC ACCCCCCTCC GGGGGTGCGC CCGGGGGCCC |
| 421 | CCGGCCCCAT GGGGCCGGTT GTTTACTCGA TCTCCACTGA CTCATTGTCC TCAAACAACT |
| 481 | TTCGACACCT GATTCCCTTG ATCTTGAAGG GTCCTGTCTC GTCTGCAATC ATAACAGATC |
| 541 | CTAGAGTCTT ACTTCTTATT ATACTAAAGT GACCACAATT CAACCAATCT TTGGCATCAT |
| 601 | GCAACATGTG TTCAAACACT TCGGGGAAAT TTTCAATCAT GAGTCTTAAA TCCTGCTCGT |
| 661 | TCATACTTAT TCCCTTGTTG TGAGACTGTG CACTTGAAAG GTACTGAAAA AGGTTGGCAA |
| 721 | TAAATCTTGG CCTTTTCTCA GGTTCTAATG CTTCCAGTGC AATGATGACC ACCTTTGAGT |
| 781 | CTAAGTTCAC TTCCAATCTA GAAACCACTC TGTTGCCCTC TTTGATCAAC CCACCCTCTA |
| 841 | AAATGAGGGG TTGCATCCCA ACATCAGGAC CAATCAACTT ATAGGAAAAT TTGTTTTTCA |
| 901 | AATCCTTGAA ACGATTTTTC AAATCTATTC TCACCTTCTG GAACACAGTT GACCTTGACT |
| 961 | TGAAGTGAAT GTCTTGACCT TCCAATAGAT CATTGAAGTC TAGAACATCT TTTCCGTTGA |
| 1021 | TGAGAGGATT CAGAACCAAA AGTGACACAC CATCCAGACT TATGTGATTC CCGGAAGATT |
| 1081 | GAGAAACATA ATACTCAACA GAATGGGGGT TCAACAATAG GTAACCATCA GAGTCCAATG |
| 1141 | AGTCCAGCAA TGACTCCCTT TCAATAAGAA ATCTTAATTT TAATATGTAA TTGGTAGACC |
| 1201 | TCTCATATCT AAATTTGTGG CTCACTCTCT TATGAGAAAA TGTTAGGTTG AGCTCAATGG |
| 1261 | GAATGACCTC AGAAGGTGAT GCTAAAATGA GTTGTTCAAT GTTCTCATAG TTATCTCTAT |
| 1321 | TCACCCAGTC AAGTTCATTA ATAAATACAC TAATGTTCAA ATTAACACAG GACAAAATCA |
| 1381 | GTTTGCTGCT TACAAAGCCA ACATCCAAGT CATCCAGATT CATTGTCCTA GAAGTGTTAT |
| 1441 | TCTTTTTGCA GTCACAAATG AACTGGGTTA ATTGTTTCAG ATCATGTTGT GCATTGTTTG |
| 1501 | GCAACAATTC AAGCTCACCA AACCAAAAAT ATTTCTTGAA CTGAGATGTT GACATAATCA |
| 1561 | CAGGCACCAA CATTGACTCA AACAAAATCT GTATCAAGAA ATTTGTGCAC ACTTCTTCTG |
| 1621 | GTTCAAGGTT GAATCCTCTC TCCAGTGGAT GAGACTCTCT GCTATGGGAC ATTGCAAGCT |
| 1681 | CATTTTGCTT TACAATATAC AATTCTTCTC TGCGATGTTT TATAATATGA CTAACAATAC |
| 1741 | CAAGACATTC TGATGTTATA TCAATTGCCA CACAAAGGTC TAAGAACTTT ATCCTCTGAA |
| 1801 | CCCATGATAG CCTCAGCATA TTCAAATCAG ACAGGAAAGG GGATATGTGT TCATCAAATA |
| 1861 | GTGTAGGGAA GTTCCTCCTG ATTGAGTAAA GTATGTGGTT GATGCCCACC TTGTCCTCAA |
| 1921 | GCTCAGAATG TGTGCTTGGT TTTATTGGCC AGAAGTGATT GGGATTGTTT AGGTGAGTGA |
| 1981 | CTATCTTGGG TACTTCAGCT TTTTGAAACA CCCAGTTACC CAACTCGCAA GCATTGGTTA |
| 2041 | ACACAAGAGC AAAATAATCC CAAATTAAGG GTCTGGAGTA CTCACTTACT TCACCAAGTG |
| 2101 | CTGCTTTACA ATAAACACCT TTGCGCTGAT TACAAAAGTG ACAATCACGG TGTAAGATAA |
| 2161 | TCTTGCTTGT AATATCCCTG ATATACTTAA ATCCTCCTTT CCCGTCTCTT ACACATTTTG |
| 2221 | AGCCCATACT TTTGCAAACT CCTATGAATC CTGATGCTAT GCTGCTCTGA AAAGCTGATT |
| 2281 | TGTTGATAGC ATCAGCCAAA ATCTTCTTAG CCCCTCTGAC ATAGTTCTTT GATAATTTGG |
| 2341 | ACTGTACGGA TTTGACAAGA CTGGGTATTT CTTCTCGCTG CACAGTTCTT GTTGTGCTCA |
| 2401 | TTAACTTAGT ACGAAGCACC AATCTGAGAT CACCATGAAC CCTTAAATTT AACCACCTAA |
| 2461 | TATTAAGAGC ATCCTCAATA GCCTCAGTCT CGACATCACA AGTCTCTAAT AACTGTTTTA |
| 2521 | AGCAGTCATC CGGTGATTGC TGAAGAGTTG TTACAATATA ACTTTCTTCC AGGGCTCCAG |
| 2581 | ACTGTATTTT GTAAAATATT TTCCTGCATG CCTTTCTGAT TATTGAAAGT AGCAGATCAT |
| 2641 | CAGGAAATAG TGTCTCAATT GATCGCTGAA GTCTGTACCC TCTCGACCCA TTAACCCAAT |
| 2701 | CGAGTACATC CATTTCTTCC AGGCACAAAA ATGGATCATT TGGAAACCCA CTATAGATTA |
| 2761 | TCATGCTATT TGTTCGTTTT GCAATGGCCC CTACAACCTC TATTGACACC CCGTTAGCAA |
| 2821 | CACATTGGTC CAGTATTGTG TCAATTGTAT CTGCTTGCTG ATTGGGTGCT TTAGCCTTTA |
| 2881 | TGTTGTGTAG AGCTGCAGCA ACAAACTTTG TAAGGAGGGG GACTTCTTGT GACCAAATGA |
| 2941 | AGAATCTCGA TTTGAACTCA CTTGCAAAGG TCCCCACAAC TGTTTTAGGG CTCACAAACT |
| 3001 | TGTTGAGTTT GTCTGATAGA AAGTAGTGAA ACTCCATACA GTCCAATACC AATTCAACAT |
| 3061 | TCAACTCATC TCTGTCCTTA AATTTGAAAC CCTCATTCAA GGATAACATG ATCTCATCAT |
| 3121 | CACTCGAAGT ATATGAGATG AACCGTGCTC CATAACAAAG CTCCAATGCG TAATTGATGA |
| 3181 | ACTGCTCAGT GATTAGACCA TATAAGTCAG AGGTGTTGTG TAGGATGCCC TGACCCATAT |
| 3241 | CTAAGACTGA AGAGATGTGT GATGGTACCT TGCCCTTCTC AAAGTACCCA AACATAAATT |
| 3301 | CCTCTGCAAT TGTGCACCCC CCTTTATCCA TCATACCCAA CCCCCTTTTC AAGAAACCTT |
| 3361 | TCATGTATGC CTCAACGACA TTGAAGGGCA CTTCCACCAT CTTGTGAATG TGCCATAGCA |
| 3421 | ATATGTTGAT GACTGCAGCA TTGGGAACTT CTGACCCATC TTTGAGTTTG AACTCAAGAC |
| 3481 | CTTTTAATAA TGCGGCAAAG ATAACCGGCG ACATGTGTGG CCCCCATTTT GAATGGTCCA |
| 3541 | TTGACACCGC AAGACCACTT TGCCTAACAA CTGACTTCAT GTCTAATAAT GCTCTCTCAA |
| 3601 | ACTCTTTCTC GTTGTTCAGA CAAGTATACC TCATGTTTTG CATAAGGGAT TCAGAGTAAT |
| 3661 | CCTCAATGAG TCTGGTTGTG AGTTTAGTAT TTAAATCACC GACATAAAGC TCCCTGTTGC |
| 3721 | CACCCACCTG TTCTTTATAA GAAAGACCAA ATTTCAATCT CCCTACATTG GTGGATACAC |
| 3781 | CAGACCTCTC TGTGGGAGAC TCATCTGAAT AGAAACAGAG ATTTCGTAAG GATGAGTTGG |
| 3841 | TAAAAAAGCT TTGATCCAAT CTTTTAGCTA TCGATTCAGA ATTGCTCTCT CTTGAGCTTA |
| 3901 | TACGTGATGT CTCTCTAATT TGTAGTGCTG CATCTGTGAA CCCAAGTCTG CTTCTACTTT |
| 3961 | TGTGATCATA TCTTCCGACT CGATTATCAT AATCGCTTGC AATGAGAATG TATTTAAAGC |
| 4021 | ACTCAAAATA ATCAGCTTCT TTGTACGCCT TCAATGTGAG GTTCTTTATT AAAAACTCCA |
| 4081 | GAGGACACGG ATTCATTAGT CTGTCTGCAA AGTACACTGA TCTAGCAGTG ACATCCTCAT |
| 4141 | AGATCAAGTT TACAAGATCC TCATACACTT CTGCTGAAAA CAGGCTGTAA TCAAAATCCT |
| 4201 | TTACATCATG AAGTGAAGTC TCTCTTTTGA TGACAACCAT TGTCGATTTG GGCCATAATC |
| 4261 | TCTCTAGTGG ACATGAAGTC TTAAGGTTGG TTTTGACATT GGTGTCAACC TTAGACAATA |
| 4321 | CTTTTGCAAC TCTGGTCTCA ATTTCTTTAA GACAGTCACC CTGATCTTCT GATAGTAACT |
| 4381 | CTTCAACTCC ATCAGGCTCT ATTGACTCCT TTTTTATTTG GATCAATGAT GACAACCTCT |
| 4441 | TCAGAATCTT GAAATTTACC TCCTTTGGAT CCAACTTGTA TTTACCCTTA GTTTTGAAAT |
| 4501 | GTTCAATCAT TTCCACAACA ACAGCAGACA CAATGGAAGA GTAATCATAT TCAGTGATGA |
| 4561 | CCTCACCAAC TTCATTGAGT TTTGGAACCA CCACACTTTT GTTGCTGGAC ATATCCAAGG |
| 4621 | CTGTACTTGT GAAGGAGGGA GTCATAGGGT CACAAGGAAG CAGGGGTTTC ACTTCCAATG |
| 4681 | AGCTACTGTT AAATAGTGAT AGACAAACAC TAAGTACATC CTTATTCAAC CCCGGCCTTC |
| 4741 | CCTCACATTT GGATTCCAGC TTTTTACCAA GTAGTCTCTC TATATCATGC ACCATCTTCT |
| 4801 | CTTCTTCCTC AGTAGGAAGT TCCATACTAT TAGAAGGGTT GACCAAGACT GAATCAAACT |
| 4861 | TTAACTTTGG TTCCAAGAAC TTCTCAAAAC ATTTGATTTG ATCAGTTAAT CTATCAGGGG |
| 4921 | TTTCTTTGGT TATAAAATGG CATAAATAGG AGACATTCAA AACAAACTTA AAGATCTTAG |
| 4981 | CCATATCTTC CTCTCTGGAG TTGCTGAGTA CCAGAAGTAT CAAATCATCA ATAAGCATTG |
| 5041 | CTGTCTGCCA TTCTGAAGGT GTTAGCATAA CGACTTTCAA TTTCTCAAAC AATTCTTTAA |
| 5101 | AATGAACTTC ATTTACAAAG GCCATAATGT AATATCTAAA GCCTTGCAAG TAAACTTGAA |
| 5161 | TACGCTTGGA AGGGGTGCAC AGTATGCAGA GAATAAGTCG TCTGAGTAAA TCAGAAACAG |
| 5221 | AATCCAAGAG GGGTTGGGAC ATAAAGTCCA ACCAGGATAA CATCTCCACA CAAGTCCTTT |
| 5281 | GAATCACATC TGCACTAAAG ATCGGTAAGA AAAATCTCTT GGGATCACAG TAAAAAGACG |
| 5341 | CTTTTGTTTC ATACAAACCC CCACTTTTGG ATCTATAAGC AACAGCATAA CACCTGGACC |
| 5401 | TCTCCCCTGT CTTCTGGTAC AGTAGTGTGA GAGAACCTCC TTCTCCAAAT CGCTGGAAGA |
| 5461 | AAACTTCGTC ACAGTAAACC TTCCCATAAA ACTCATCAGC ATTGTTCACC TTCATCTTAG |
| 5521 | GAACTGCTGC TGTCTTCATG CTATTAATGA GTGACAAACT CAAACTTGAC AATGTTTTCA |
| 5581 | GCAATTCCTC AAACTCACTT TCGCCCATGA TGGTATAATC AGGCTGCCCT CTTCCTGGCC |
| 5641 | TACCCCCACA CATACACTGT GACTTTGTCT TGTATTGAAG ACAGGGTTTA GCACCCCATT |
| 5701 | CATCTAACAC TGATGTTTTC AGATTGAAGT AATATTCAAC ATCAGGTTCC CGTAGAAGAG |
| 5761 | GGAGAATGTC ATCAAGGGGA AGTTCACCAC AGACCGAGCT CAGTCTCTTC TTAGCCTTCT |
| 5821 | CTAACCAGTT GGGGTTTTTA ATGAATTTTT TAGTGATTTG TTCCATCAGG AAGTCGACAT |
| 5881 | TAATCAACCT GTCATTTACA GACGGTAACC CTTGCATTAG GAGCACCTCT CTGAACACAG |
| 5941 | CACCTGGAGA AGACTTGTCC AAGTCACACA AAATGTTGTA CATGATAAGG TCCAGAACCA |
| 6001 | ACATGGTGTT CCTCCTTGTG TTAAAAACCT TTTGAGACTT AATTTTGTTG CATATTGAAA |
| 6061 | GTACTCTAAA ATATTCTCTG CTTTCAGTTG ATGAATGCTT GACCTCAGAT TGCCTGAGTT |
| 6121 | GGCCTATTAT GCCCAAAATG TGTACTGAGC AAAACTCACA TAATCTGATT TCTGATTTAG |
| 6181 | GTACATCTTT GACAGAACAT TGGATAAATT CATGGTTCTG AAGTCTAGAA ATCATATCTT |
| 6241 | CCCTATCTGT AGCCTGCAGT TTCCTATCGA GTTGACCAGC AAGTTGCAAC ATTTTAAATT |
| 6301 | GCTGAAAGAT TTCCATGATT TTTGTTCTAC ATTGATCTGT TGTCAGTTTA TTATTAATGC |
| 6361 | CAGACATTAA TGCCTTTTCC AACCTCACTT TGTAAGGAAG TCCCCTTTCC TTTACAGCAA |
| 6421 | GTAGTGACTC CAGACCGAGA CTCTGATTTT CTAAGGATGA GAGGGAACTT ATAAGGCGTT |
| 6481 | CGTACTCCAA CTCCTCAACT TCTTCACCAG ATGTCCTTAA TCCATCCATG AGTTTTAAAA |
| 6541 | GCAACCACCG AAGTCTCTCT ACCACCCAAT CAGGAACAAA TTCTACATAA TAACTGGATC |
| 6601 | TACCGTCAAT AACAGGTACT AAGGTTATGT TCTGTCTCTT GAGATCAGAA CTAAGCTGCA |
| 6661 | ACAGCTTCAA AAAGTCCTGG TTGTATTTCT TCTCAAATGC TTCTTGACTG GTCCTCACAA |
| 6721 | ACACTTCCAA AAGAATGAGG ACATCTCCAA CCATACAGTA ACCATCTGGT GTAACATCCG |
| 6781 | GCAATGTAGG ACATGTTACT CTCAACTCCC TAAGGATAGC ATTGACAGTC ATCTTTGTGT |
| 6841 | TGTGTTTGCA GGAGTGTTTC TTGCATGAAT CCACTTCCAC TAGCATGGAC AAAAGCTTCA |
| 6901 | GGCCCTCTAT CGTGATGGCC CTATCTTTGA CTTGTGCAAG AACGTTGTTT TTCTGTTCAG |
| 6961 | ATAGCTCTTC CCATTCGGGA ACCCATTTTC TGACTATGTC TTTAAGTTCG AAAACGTATT |
| 7021 | CCTCCATGAT CAAGAAATGC CTAGGATCCT CGGTGCG |
| SEQ ID NO: 22: | |
| 1 | CGCACCGGGG ATCCTAGGCA TACCTTGGAC GCGCATATTA CTTGATCAAA GATGGGACAA |
| 61 | GTTGTGACTT TGATCCAGTC TATACCCGAA GTCCTGCAGG AGGTCTTCAA TGTCGCCTTA |
| 121 | ATCATTGTCT CAACCCTATG CATCATCAAA GGATTTGTCA ATCTGATGAG ATGTGGCCTA |
| 181 | TTCCAACTCA TCACCTTCCT CATTTTGGCT GGCAGAAGTT GTGATGGCAT GATGATTGAT |
| 241 | AGGAGGCACA ATCTCACCCA CGTTGAGTTC AACCTCACAA GAATGTTTGA CAACTTGCCA |
| 301 | CAATCATGTA GCAAGAACAA CACACATCAT TACTACAAAG GACCATCTAA CACAACATGG |
| 361 | GGAATTGAAC TCACTTTGAC AAACACATCC ATTGCAAATG AAACTACTGG AAACTTTTCC |
| 421 | AACATCAGAA GCCTTGCATA TGGTAACATT AGTAATTGTG ATAAGACAGA AGAAGCAGGT |
| 481 | CACACATTAA AATGGTTGCT TAATGAGTTA CACTTCAATG TGCTCCATGT CACTCGTCAT |
| 541 | GTAGGTGCCA GATGCAAAAC AGTTGAGGGT GCTGGGGTGT TGATCCAGTA CAACTTGACA |
| 601 | GTTGGGGATA GAGGAGGTGA GGTTGGCAGA CATCTTATTG CGTCGCTTGC TCAAATCATT |
| 661 | GGGGACCCAA AAATTGCGTG GGTTGGAAAA TGTTTCAATA ACTGTAGTGG AGGGTCTTGC |
| 721 | AGACTAACAA ACTGTGAAGG TGGGACACAT TACAATTTCC TGATCATACA GAACACCACA |
| 781 | TGGGAAAATC ACTGTACATA TACTCCAATG GCAACAATAA GGATGGCTCT CCAAAAAACT |
| 841 | GCTTATAGTT CTGTGAGCAG GAAACTCCTT GGCTTTTTCA CTTGGGACTT GAGTGACTCT |
| 901 | ACTGGGCAAC ATGTCCCAGG TGGTTACTGT TTGGAGCAAT GGGCTATTGT TTGGGCTGGA |
| 961 | ATAAAATGTT TTGATAACAC TGTGATGGCA AAATGCAACA AAGATCACAA TGAAGAATTT |
| 1021 | TGCGATACGA TGAGGTTATT TGATTTCAAT CAGAATGCTA TCAAAACCTT ACAACTTAAT |
| 1081 | GTTGAGAATT CGTTGAATCT CTTTAAAAAG ACTATCAACG GACTTATTTC TGACTCACTT |
| 1141 | GTGATTAGAA ACAGTCTCAA ACAGCTTGCC AAAATCCCTT ATTGCAACTA TACAAAATTT |
| 1201 | TGGTACATCA ATGATACCAT CACAGGAAGA CATTCTTTAC CGCAGTGTTG GTTAGTTCAC |
| 1261 | AATGGCTCGT ACCTCAATGA AACGCATTTT AAGAATGATT GGTTGTGGGA GAGCCAGAAT |
| 1321 | CTGTACAATG AAATGCTGAT AAAAGAATAT GAAGAAAGAC AAGGTAAGAC TCCACTAGCA |
| 1381 | TTGACAGACA TTTGCTTCTG GTCTTTGGTG TTTTACACCA TCACAGTGTT TCTCCACTTA |
| 1441 | GTTGGAATAC CCACTCATAG GCACATCATT GGTGATGGCT GTCCGAAGCC ACATAGGATT |
| 1501 | ACTAGGAACT CTCTTTGCAG CTGTGGGTAT TATAAAATCC CAAAGAAACC CTACAAATGG |
| 1561 | GTGAGACTGG GTAAATAAGC CCTAGCCTCG ACATGGGCCT CGACGTCACT CCCCAATAGG |
| 1621 | GGAGTGACGT CGAGGCCTCT GAGGACTTGA GCTCAGAGGT TGATCAGATC TGTGTTGTTC |
| 1681 | CTGTACAGCG TGTCAATAGG CAAGCATCTC ATCGGCTTCT GGTCCCTAAC CCAGCCTGTC |
| 1741 | ACTGTTGCAT CAAACATGAT GGTATCAAGC AATGCACAGT GAGGATTCGC AGTGGTTTGT |
| 1801 | GCAGCCCCCT TCTTCTTCTT CTTTATGACC AAACCTTTAT GTTTGGTGCA GAGTAGATTG |
| 1861 | TATCTCTCCC AGATCTCATC CTCAAAGGTG CGTGCTTGCT CGGCACTGAG TTTCACGTCA |
| 1921 | AGCACTTTTA AGTCTCTTCT CCCATGCATT TCGAACAAAC TGATTATATC ATCTGAACCT |
| 1981 | TGAGCAGTGA AAACCATGTT TTGAGGTAAA TGTCTGATGA TTGAGGAAAT CAGGCCTGGT |
| 2041 | TGGGCATCAG CCAAGTCCTT TAAAAGAAGA CCATGTGAGT ACTTGCTTTG CTCTTTGAAG |
| 2101 | GACTTCTCAT CGTGGGGAAA TCTGTAACAA TGTATGTAGT TGCCCGTGTC AGGCTGGTAG |
| 2161 | ATGGCCATTT CCACCGGATC ATTTGGTGTT CCTTCAATGT CAATCCATGT GGTAGCTTTT |
| 2221 | GAATCAAGCA TCTGAATTGA GGACACAACA GTGTCTTCTT TCTCCTTAGG GATTTGTTTA |
| 2281 | AGGTCCGGTG ATCCTCCGTT TCTTACTGGT GGCTGGATAG CACTCGGCTT CGAATCTAAA |
| 2341 | TCTACAGTGG TGTTATCCCA AGCCCTCCCT TGAACTTGAG ACCTTGAGCC AATGTAAGGC |
| 2401 | CAACCATCCC CTGAAAGACA AATCTTGTAT AGTAAATTTT CATAAGGATT TCTCTGTCCG |
| 2461 | GGTGTAGTGC TCACAAACAT ACCTTCACGA TTCTTTATTT GCAATAGACT CTTTATGAGA |
| 2521 | GTACTAAACA TAGAAGGCTT CACCTGGATG GTCTCAAGCA TATTGCCACC ATCAATCATG |
| 2581 | CAAGCAGCTG CTTTGACTGC TGCAGACAAA CTGAGATTGT ACCCTGAGAT GTTTATGGCT |
| 2641 | GATGGCTCAT TACTAATGAT TTTTAGGGCA CTGTGTTGCT GTGTGAGTTT CTCTAGATCT |
| 2701 | GTCATGTTCG GGAACTTGAC AGTGTAGAGC AAACCAAGTG CACTCAGCGC TTGGACAACA |
| 2761 | TCATTAAGTT GTTCACCCCC TTGCTCAGTC ATACAAGCGA TGGTTAAGGC TGGCATTGAT |
| 2821 | CCAAATTGAT TGATCAACAA TGTATTATCC TTGATGTCCC AGATCTTCAC AACCCCATCT |
| 2881 | CTGTTGCCTG TGGGTCTAGC ATTAGCGAAC CCCATTGAGC GAAGGATTTC GGCTCTTTGT |
| 2941 | TCCAACTGAG TGTTTGTGAG ATTGCCCCCA TAAACACCAG GCTGAGACAA ACTCTCAGTT |
| 3001 | CTAGTGACTT TCTTTCTTAA CTTGTCCAAA TCAGATGCAA GCTCCATTAG CTCCTCTTTG |
| 3061 | GCTAAGCCTC CCACCTTAAG CACATTGTCC CTCTGGATTG ATCTCATATT CATCAGAGCA |
| 3121 | TCAACCTCTT TGTTCATGTC TCTTAACTTG GTCAGATCAG AATCAGTCCT TTTATCTTTG |
| 3181 | CGCATCATTC TTTGAACTTG AGCAACTTTG TGAAAGTCAA GAGCAGATAA CAGTGCTCTT |
| 3241 | GTGTCCGACA ACACATCAGC CTTCACAGGA TGGGTCCAGT TGGATAGACC CCTCCTAAGG |
| 3301 | GACTGTACCC AGCGGAATGA TGGGATGTTG TCAGACATTT TGGGGTTGTT TGCACTTCCT |
| 3361 | CCGAGTCAGT GAAGAAGTGA ACGTACAGCG TGATCTAGAA TCGCCTAGGA TCCACTGTGC |
| 3421 | G |

As used herein, reference to a polynucleotide as described herein and/or reference to the nucleic acid sequence of one or more SEQ ID NOs can include polynucleotides having a sequence identity of at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to an identified reference polynucleotide sequence.

In this context, “sequence identity” refers to the identity between two polynucleotide sequences. Sequence identity is generally determined by aligning the bases of the two polynucleotides (for example, aligning the nucleotide sequence of the candidate sequence and a nucleotide sequence that includes, for example, a nucleotide sequence that encodes a protein of SEQ ID NO:1, 2, 3, or 4) to optimize the number of identical nucleotides along the lengths of their sequences; gaps in either or both sequences are permitted in making the alignment in order to optimize the number of shared nucleotides, although the nucleotides in each sequence must nonetheless remain in their proper order. A candidate sequence is the sequence being compared to a known sequence—e.g., a nucleotide sequence that includes the appropriate nucleotide sequence selected from, for example, SEQ ID NO:21 or 222. For example, two polynucleotide sequences can be compared using the Blastn program of the BLAST 2 search algorithm, as described by Tatiana et al., FEMS Microbiol Let., 1999; 174: 247-250, and available on the world wide web at ncbi.nlm.nih.gov/BLAST/. The default values for all BLAST 2 search parameters may be used, including reward for match=1, penalty for mismatch=−2, open gap penalty=5, extension gap penalty=2, gap x_dropoff=50, expect=10, wordsize=11, and filter on.

Optionally and preferably, the second genomic segment and third genomic segment each independently include one or more additional coding regions that include an open reading frame encoding one or more Mtb proteins. The one or more additional coding regions present on a genomic segment can be expressed as a monocistronic mRNA or a polycistronic mRNA. A monocistronic mRNA includes one open reading frame. As described herein, the open reading frame can encode one or more proteins. A polycistronic mRNA is one that includes two or more open reading frames, where each open reading frame encodes a protein. A genomic segment can include one or more coding regions that are expressed as a monocistronic mRNA molecule, one or more coding regions that are expressed as a polycistronic mRNA molecule, or a combination of coding regions where one or more are expressed as a monocistronic mRNA and one or more are expressed as a polycistronic mRNA. In one embodiment, a genomic segment includes, an open reading frame encoding a Z protein, L RdRp protein, NP protein, or glycoprotein, and one coding region that is expressed as a monocistronic mRNA molecule that encodes two or more proteins.

In one embodiment, a monocistronic message includes an open reading frame that encodes a protein. In another embodiment, a monocistronic message includes separate regions that encode proteins, but each separate region does not include a translation start codon at its 5′ end and a translation stop codon at its 3′ end. Instead, the nucleotides between the separate regions that encode proteins encode a self-cleaving peptide. An example of a class of self-cleaving peptide is the 2A peptide. 2A peptides are amino acid sequences that can result in a molecular event after translation that has the effect of cleaving the amino acid sequence. Four examples of 2A peptides are shown on Table 1. The cleavage is trigged by breaking a peptide bond between the Proline (P) and Glycine (G) in the C-terminal end of the 2A peptide. The molecular mechanism of 2A-peptide-mediated cleavage is unknown but is believed to involve ribosomal “skipping” of glycyl-prolyl peptide bond formation rather than true proteolytic cleavage (Wang et al., 2015, Scientific Reports. 5 (1), doi:10.1038/srep16273, ISSN 2045-2322; Ryan et al., 2001, Journal of General Virology. 82(5):1013-1025; and Sharma et al., 2012, Nucleic Acids Research. 40(7):3143-3151).

TABLE 1
2A peptides

| Peptide | Amino acid sequence¹ |
|---|---|
| T2A | (GSG)EGRGSLLTCGDVEENPGP |
| | (SEQ ID NO: 8) |
| P2A | (GSG)ATNFSLLKQAGDVEENPGP |
| | (SEQ ID NO: 9) |
| E2A | (GSG)QCTNYALLKLAGDVESNPGP |
| | (SEQ ID NO: 10) |
| F2A | (GSG)VKQTLNFDLLKLAGDVESNPGP |
| | (SEQ ID NO: 11) |

¹Addition of the GSG residues at the 5′ end of the peptide is optional and can improve cleavage efficiency.

In one embodiment, a polycistronic message includes separate open reading frames, e.g., one or more open reading frames within a polycistronic message each having a translation start codon at its 5′ end and a translation stop codon at its 3′ end. Each coding region is separately translated beginning at the translation start codon and ending at the translation stop codon. An intercistronic region can be located between two of the coding regions. An intercistronic region is at least one nucleotide that is not translated into a protein.

In one embodiment, the number of Mtb proteins encoded by a genomic segment can be 1, 2, 3, 4, 5, 6, 7, 8, 9, 10, or more. A factor that influences the number of coding regions is the total length of the genomic segments. As described herein, in some embodiments it is useful to rescue virus and produce an infectious virus particle. In one embodiment, when the second and/or third genomic segments include one or more additional coding regions encoding one or more proteins, the maximum size in nucleotides of the added coding region(s) to a genomic segment is no greater than 2 kilobases (kb), no greater than 1.8 kb, or no greater than 1.6 kb.

A Mtb protein useful herein results in a humoral immune response, a cell-mediated immune response, or a combination thereof when expressed in a subject. In one embodiment, the protein is at least 6 amino acids in length. The nucleotide sequence encoding a Mtb protein can be modified to reflect the codon usage bias of a cell in which the protein will be expressed. The usage bias of nearly all cells in which a Pichinde virus would be expressed is known to the skilled person.

Examples of Mth proteins that can be expressed include the acute-phase infection proteins Ag85B, ESAT-6 (also referred to as EsxA), and EsxH. One example of an Ag85B protein from Mtb is the sequence available at Genbank accession number NP 216402 (SEQ ID NO:5). One example of an ESAT-6 (EsxA) protein from Mtb is the sequence available at Genbank accession number YP_178023 (SEQ ID NO:6). One example of an EsxH protein from Mtb is the sequence available at Genbank accession number NP_214802 (SEQ ID NO:7).

Examples of Mtb proteins that can be expressed include the latency phase protein Rv1733. One example of a Rv1733 protein from Mtb is GenBank accession number NP_216249 or the Mtb protein derived from the nucleotides 259-1359 of Genbank accession number KY611402 (SEQ ID NO:19).

Examples of Mtb proteins that can be expressed include the RpfA protein expressed during the resuscitation phase. One example of a RpfA protein from Mtb is GenBank accession number NP_215382 or the Mtb protein derived from nucleotides 257 to 1477 of Genbank accession number KY611403 (SEQ ID NO:20).

Other examples of Ag85B, ESAT-6(EsxA), EsxH, Rv1733, and RpfA proteins include proteins having structural similarity with SEQ ID NO:5, 6, 7, 19, or 20, respectively. In one embodiment, an Ag85B, ESAT-6(EsxA), EsxH, Rv1733, and/or RpfA protein includes one or more substitutions. In one embodiment, one or more of the substitutions is a conservative substitution. Pichinde virus is an arenavirus, and one characteristic of an arenavirus is an ambisense genome. As used herein, “ambisense” refers to a genomic segment having both positive sense and negative sense portions and coding strategies. For example, the first genomic segment of a Pichinde virus described herein is ambisense, encoding a Z protein in the positive sense and encoding a L RdRp protein in the negative sense. Thus, one of the two coding regions of the first genomic segment is in a positive-sense orientation and the other is in a negative-sense orientation. When the second and/or the third genomic segment includes a second coding region encoding a protein, the coding region encoding the protein is in a negative-sense orientation compared to the NP protein of the second genomic segment and to the glycoprotein of the third genomic segment.

Each genomic segment also includes nucleotides encoding a 5′ untranslated region (UTR) and a 3′ UTR. These UTRs are located at the ends of each genomic segment. Nucleotides useful as 5′ UTRs and 3′ UTRs include those present in Pichinde virus and are readily available to the skilled person (see, for instance, Buchmeier et al., 2007, Arenaviridae: the viruses and their replication. In: Knipe and Howley (eds), Fields Virology. 5th ed.

Philadelphia, PA: Lippincott Williams & Wilkins. pp. 1791-1827). In one embodiment, a genomic segment that encodes a Z protein and an L RdRp protein includes a 5′ UTR sequence that is 5′ CGCACCGGGGAUCCUAGGCAUCUUUGGGUCACGCUUCAAAUUUGUCCAAUU UGAACCCAGCUCAAGUCCUGGUCAAAACUUGGG (SEQ ID NO:12) and a 3′ UTR sequence that is CGCACCGAGGAUCCUAGGCAUUUCUUGAUC (SEQ ID NO:13). In one embodiment, a genomic segment that encodes a NP protein or a glycoprotein includes a 5′ UTR sequence that is 5′ CGCACCGGGGAUCCUAGGCAUACCUUGGACGCGCAUAUUACUUGAUCAAAG (SEQ ID NO:14) and a 3′ UTR sequence that is 5′ CGCACAGUGGAUCCUAGGCGAUUCUAGAUCACGCUGUACGUUCACUUCUUCA CUGACUCGGAGGAAGUGCAAACAACCCCAAA (SEQ ID NO:15). Alterations in these sequences are permitted, and the terminal 27-30 nucleotides are highly conserved between the genomic segments.

Each genomic segment also includes an intergenic region (IGR) located between the coding region encoding a Z protein and the coding region encoding a L RdRp protein, between the coding region encoding a nucleoprotein and the additional coding region(s), and between the coding region encoding a glycoprotein and the additional coding region(s). Nucleotides useful as an intergenic region are those present in Pichinde virus and are readily available to the skilled person. In one embodiment, an IGR sequence of a genomic segment that encodes a Z protein and an L RdRp protein includes 5′ ACCAGGGCCCCUGGGCGCACCCCCCUCCGGGGGUGCGCCCGGGGGCCCCCGG CCCCAUGGGGCCGGUUGUU (SEQ ID NO:16). In one embodiment, an IGR sequence of a genomic segment that encodes a NP protein or a glycoprotein includes 5′ GCCCUAGCCUCGACAUGGGCCUCGACGUCACUCCCCAAUAGGGGAGUGACGU CGAGGCCUCUGAGGACUUGAGCU (SEQ ID NO:17).

In one embodiment, a coding region can include nucleotides that encode a protein that is useful as a detectable marker, e.g., a molecule that is easily detected by various methods.

Examples include fluorescent proteins (e.g., green, yellow, blue, or red fluorescent proteins), luciferase, chloramphenicol acetyl transferase, and other molecules (such as c-myc, flag, 6×his, HisGln (HQ) metal-binding peptide, and V5 epitope) detectable by their fluorescence, enzymatic activity or immunological properties.

### Vectors

One or more of the genomic segments described herein can be present in a vector. For instance, all genomic segments can be present in one vector, two can be present in one vector, or each genomic segment is present in different vectors. In one embodiment, the sequence of a genomic segment in the vector is antigenomic, and in one embodiment the sequence of a genomic segment in the vector is genomic. As used herein, “anti-genomic” refers to a genomic segment that encodes a protein in the orientation opposite to the viral genome. For example, Pichinde virus is a negative-sense RNA virus. However, each genomic segment is ambisense, encoding proteins in both the positive-sense and negative-sense orientations. “Anti-genomic” refers to the positive-sense orientation, while “genomic” refers to the negative-sense orientation.

A vector is a replicating polynucleotide, such as a plasmid, phage, or cosmid, to which another polynucleotide may be attached so as to bring about the replication of the attached polynucleotide. Construction of vectors containing a genomic segment, and construction of genomic segments including insertion of a polynucleotide encoding an protein, employs standard ligation techniques known in the art. See, e.g., Sambrook et al, Molecular Cloning: A Laboratory Manual., Cold Spring Harbor Laboratory Press (1989) or Ausubel, R. M., ed. Current Protocols in Molecular Biology (1994). A vector can provide for further cloning (amplification of the polynucleotide), i.e., a cloning vector, or for expression of an RNA encoded by the genomic segment, i.e., an expression vector. The term vector includes, but is not limited to, plasmid vectors, viral vectors, cosmid vectors, or artificial chromosome vectors. Typically, a vector is capable of replication in a prokaryotic cell and/or a eukaryotic cell. In one embodiment, the vector replicates in prokaryotic cells, and not in eukaryotic cells. In one embodiment, the vector is a plasmid.

Selection of a vector depends upon a variety of desired characteristics in the resulting construct, such as a selection marker, vector replication rate, and the like. Suitable host cells for cloning or expressing the vectors herein are prokaryote or eukaryotic cells.

An expression vector optionally includes regulatory sequences operably linked to the genomic segment. The term “operably linked” refers to a juxtaposition of components such that they are in a relationship permitting them to function in their intended manner. A regulatory sequence is “operably linked” to a genomic segment when it is joined in such a way that expression of the genomic segment is achieved under conditions compatible with the regulatory sequence. One regulatory sequence is a promoter, which acts as a regulatory signal that bind RNA polymerase to initiate transcription of the downstream (3′ direction) genomic segment. The promoter used can be a constitutive or an inducible promoter. The invention is not limited by the use of any particular promoter, and a wide variety of promoters are known. In one embodiment, a T7 promoter is used. Another regulatory sequence is a transcription terminator located downstream of the genomic segment. Any transcription terminator that acts to stop transcription of the RNA polymerase that initiates transcription at the promoter may be used. In one embodiment, when the promoter is a T7 promoter, a T7 transcription terminator is also used. In one embodiment, a ribozyme is present to aid in processing an RNA molecule. A ribozyme may be present after the sequences encoding the genomic segment and before a transcription terminator. An example of a ribozyme is a hepatitis delta virus ribozyme. One example of a hepatitis delta virus ribozyme is 5′ AGCUCUCCCUUAGCCAUCCGAGUGGACGACGUCCUCCUUCGGAUGCCCAGGU CGGACCGCGAGGAGGUGGAGAUGCCAUGCCGACCC (SEQ ID NO:18).

Transcription of a genomic segment present in a vector results in an RNA molecule. When each of the three genomic segments is present in a cell the coding regions of the genomic segments are expressed and viral particles that contain one copy of each of the genomic segments are produced. The three genomic segments of the reverse genetics system described herein are based on Pichinde virus, an arenavirus with a segmented genome of two single-stranded ambisense RNAs. While the ability of the reverse genetics system to replicate and produce infectious virus typically requires the presence of the ambisense RNAs in a cell, the genomic segments described herein also include the complement thereof (i.e., complementary RNA), and the corresponding DNA sequences of the two RNA sequences.

A polynucleotide used to transform a host cell optionally includes one or more marker sequences, which typically encode a molecule that inactivates or otherwise detects or is detected by a compound in the growth medium. For example, the inclusion of a marker sequence can render the transformed cell resistant to an antibiotic, or it can confer compound-specific metabolism on the transformed cell. Examples of a marker sequence are sequences that confer resistance to kanamycin, ampicillin, chloramphenicol, tetracycline, and neomycin.

### Compositions

Also provided are compositions including a viral particle described herein, or the three genomic segments described herein. Such compositions typically include a pharmaceutically acceptable carrier. As used herein “pharmaceutically acceptable carrier” includes saline, solvents, dispersion media, coatings, antibacterial and antifungal agents, isotonic and absorption delaying agents, and the like, compatible with pharmaceutical administration. Additional active compounds can also be incorporated into the compositions.

A composition described herein may be referred to as a vaccine. The term “vaccine” as used herein refers to a composition that, upon administration to an animal, will increase the likelihood the recipient mounts an immune response to an protein encoded by one of the genomic segments described herein.

A composition may be prepared by methods well known in the art of pharmaceutics. In general, a composition can be formulated to be compatible with its intended route of administration. Administration may be systemic or local. Examples of routes of administration include parenteral (e.g., intravenous, intradermal, subcutaneous, intraperitoneal, intramuscular), enteral (e.g., oral), and topical (e.g., epicutaneous, inhalational, transmucosal) administration. Appropriate dosage forms for enteral administration of the compound of the present invention may include tablets, capsules or liquids. Appropriate dosage forms for parenteral administration may include intravenous administration. Appropriate dosage forms for topical administration may include nasal sprays, metered dose inhalers, dry-powder inhalers or by nebulization.

Solutions or suspensions can include the following components: a sterile diluent such as water for administration, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; electrolytes, such as sodium ion, chloride ion, potassium ion, calcium ion, and magnesium ion, and agents for the adjustment of tonicity such as sodium chloride or dextrose. pH can be adjusted with acids or bases, such as hydrochloric acid or sodium hydroxide. A composition can be enclosed in ampoules, disposable syringes or multiple dose vials made of glass or plastic.

Compositions can include sterile aqueous solutions (where water soluble) or dispersions and sterile powders for the extemporaneous preparation of sterile solutions or dispersions. For intravenous administration, suitable carriers include physiological saline, bacteriostatic water, phosphate buffered saline (PBS), and the like. A composition is typically sterile and, when suitable for injectable use, should be fluid to the extent that easy syringability exists.

It should be stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile solutions can be prepared by incorporating the active compound (e.g., a viral particle described herein) in the required amount in an appropriate solvent with one or a combination of ingredients enumerated above, followed by filtered sterilization. Generally, dispersions are prepared by incorporating the active compound into a sterile vehicle, which contains a basic dispersion medium and any other appropriate ingredients. In the case of sterile powders for the preparation of sterile injectable solutions, preferred methods of preparation include vacuum drying and freeze-drying which yields a powder of the active ingredient plus any additional desired ingredient from a previously sterilized solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the active compounds can be delivered in the form of an aerosol spray from a pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated can be used in the formulation. Such penetrants are generally known in the art, and include, for example, for transmucosal administration, detergents, bile salts, and fusidic acid derivatives. Transmucosal administration can be intranasal, for instance, accomplished through the use of droplets or nasal sprays. For transdermal administration, the active compounds are formulated into ointments, salves, gels, or creams as generally known in the art.

The active compounds may be prepared with carriers that will protect the compound against rapid elimination from the body, such as a controlled release formulation, including implants. Biodegradable, biocompatible polymers can be used, such as ethylene vinyl acetate, polyanhydrides, polyglycolic acid, collagen, polyorthoesters, and polylactic acid. Such formulations can be prepared using standard techniques. Liposomal suspensions can also be used as pharmaceutically acceptable carriers. These can be prepared according to methods known to those skilled in the art.

The data obtained from cell culture assays and animal studies can be used in formulating a range of dosage for use in an animal. The dosage of such compounds lies preferably within a range of circulating concentrations that include the ED₅₀ (the dose therapeutically effective in 50% of the population) with little or no toxicity. The dosage may vary within this range depending upon the dosage form employed and the route of administration utilized.

The compositions can be administered once to result in an immune response, or one or more additional times as a booster to potentiate the immune response and increase the likelihood immunity to the proteins is long-lasting. The skilled artisan will appreciate that certain factors may influence the dosage and timing required to effectively treat a subject, including but not limited to the severity of the disease or disorder, previous treatments, the general health and/or age of the subject, and other diseases present.

### Methods of Use

Also provided herein are methods for using the genomic segments. In one embodiment, a method includes making an infectious viral particle. Such a method includes, but is not limited to, providing a cell that includes each of the three genomic segments described herein (a first genomic segment, a second genomic segment, and a third genomic segment) and incubating the cell under conditions suitable for generating full-length genomic RNA molecules of each genomic segment. The full-length genomic RNA of each genomic segment is antigenomic.

Production of full-length genomic RNA molecules of each genomic segment results in transcription and translation of each viral gene product and amplification of the viral genome to generate infectious progeny virus particles. As used herein, an “infectious virus particle” refers to a virus particle that can interact with a suitable eukaryotic cell, such as a mammalian cell (e.g., a murine cell or a human cell) or an avian cell, to result in the introduction of the three genomic segments into the cell, and the transcription of the three genomic segments in the cell. The method can also include introducing into the cell vectors that encode the three genomic segments. Infectious virus particles are released into a supernatant and may be isolated and amplified further by culturing on a eukaryotic cell, such as, but not limited to, baby hamster kidney (BHK21) epithelial cells or African green monkey epithelial (VERO) cells. The method may include isolating a viral particle from a cell or a mixture of cells and cellular debris. The method may include inactivating virus particles using standard methods, such a hydrogen peroxide treatment. Also provided is a viral particle, infectious or inactivated, that contains three genomic segments described herein.

In one embodiment, a method includes expression of one or more Mtb proteins in a cell. Such a method includes, but is not limited to, introducing into a cell the three genomic segments described herein. In one embodiment, the introducing is by introduction of a virus particle that is infectious or inactivated. The second and/or the third genomic segment may include one or more additional coding regions encoding a Mtb protein. More than one type of virus particle may be administered. For instance, two populations of virus particles may be administered where each population encodes different proteins. The cell is a suitable eukaryotic cell, such as a mammalian cell (e.g., a murine cell or a human cell) or an avian cell. In one embodiment, the avian cell is a chicken embryonic fibroblast. The cell may be ex vivo or in vivo. The three genomic segments may be introduced by contacting a cell with an infectious virus particle that contains the three genomic segments, or by introducing into the cell vectors that include the genomic segments. The method further includes incubating the cell under conditions suitable for expression of the coding regions present on the three genomic segments.

In one embodiment, a method includes immunizing an animal. Such a method includes, but is not limited to, administering to an animal a viral particle that is infectious or inactivated, that contains the three genomic segments described herein. More than one type of virus particle may be administered. For instance, two populations of virus particles may be administered where each population encodes different Mtb proteins. The animal may be any animal in need of immunization, including a vertebrate, such as a mammal. The animal can be, for instance, a mouse, a guinea pig, a rabbit, a non-human primate, or a human. In one embodiment, the animal is an animal at risk of exposure to Mtb. In another embodiment, the animal is an animal that has a Mtb infection. The immune response may be a humoral response (e.g., the immune response includes production of antibody in response to an antigen), a cellular response (e.g. the activation of phagocytes, antigen-specific cytotoxic T-lymphocytes, and the release of cytokines in response to an antigen), or a combination thereof.

In another embodiment, a method includes treating a Mtb infection in an animal. As used herein, the term “infection” refers to the presence of and multiplication of Mtb in the body of a subject. The infection can be clinically inapparent, or result in signs associated with tuberculosis. The infection can be at an early stage, or at a late stage. In another embodiment, a method includes treating one or more symptoms of tuberculosis in an animal. In one embodiment, the method includes administering an effective amount of a composition described herein to an animal having or at risk of having aMtb infection and determining whether the amount of Mtb in the animal decreases. In one embodiment, the method includes administering an effective amount of a composition described herein to an animal having at least one sign of tuberculosis, and determining whether at least one sign of the condition is reduced.

Treatment of a sign associated with tuberculosis can be prophylactic or, alternatively, can be initiated after the development of tuberculosis. As used herein, the term “sign” refers to objective evidence in a subject of a condition caused by infection by disease. Signs associated with tuberculosis and the evaluations of such signs is routine and known in the art. Treatment that is prophylactic, for instance, initiated before a subject manifests signs of tuberculosis, is referred to herein as treatment of a subject that is “at risk” of developing tuberculosis. Accordingly, administration of a composition can be performed before, during, or after the occurrence of tuberculosis. Treatment initiated after the development of tuberculosis may result in decreasing the severity of the signs of tuberculosis, or completely removing the signs. In this aspect of the invention, an “effective amount” is an amount effective to prevent the manifestation of signs of tuberculosis, decrease the severity of the signs of tuberculosis, and/or completely remove the signs.

Also provided herein is a kit for immunizing an animal. The kit includes viral particles as described herein, where the second and/or third genomic segments each independently include a coding region that encodes one or more Mtb proteins described herein, in a suitable packaging material in an amount sufficient for at least one immunization. In one embodiment, the kit may include more than one type of viral particle, e.g., the kit may include one viral particle that encodes one or two antigens and a second viral particle that encodes one or two other antigens. Optionally, other reagents such as buffers and solutions needed to practice the invention are also included. Instructions for use of the packaged viral particles are also typically included.

As used herein, the phrase “packaging material” refers to one or more physical structures used to house the contents of the kit. The packaging material is constructed by known methods, preferably to provide a sterile, contaminant-free environment. The packaging material has a label which indicates that the viral particles can be used for immunizing an animal. In addition, the packaging material contains instructions indicating how the materials within the kit are employed to immunize an animal. As used herein, the term “package” refers to a solid matrix or material such as glass, plastic, paper, foil, and the like, capable of holding within fixed limits viral particles. Thus, for example, a package can be a glass vial used to contain an appropriate amount of viral particles. “Instructions for use” typically include a tangible expression describing the amount of viral particles, route of administration, and the like.

The invention is defined in the claims. However, below there is provided a non-exhaustive listing of non-limiting exemplary embodiments. Any one or more of the features of these embodiments may be combined with any one or more features of another example, embodiment, or aspect described herein.

### EXEMPLARY EMBODIMENTS

Embodiment 1 is a genetically engineered Pichinde virus comprising: three ambisense genomic segments, wherein the first genomic segment comprises a coding region encoding a Z protein and a coding region encoding a L RdRp protein, wherein the second genomic segment comprises a coding region encoding a nucleoprotein (NP) and a second coding region, wherein the second coding region encodes at least one Mtb acute-phase protein, at least one Mtb latency phase protein, at least one Mtb protein expressed during the resuscitation phase, or a combination thereof, and wherein the third genomic segment comprises a coding region encoding a glycoprotein and a third coding region, wherein the third coding region encodes at least one Mtb acute-phase protein, at least one Mtb latency phase protein, at least one Mtb protein expressed during the resuscitation phase, or a combination thereof.

Embodiment 2 is the virus of Embodiment 1 wherein the at least one Mtb protein encoded by the second coding region is different than the at least one Mtb protein encoded by the third coding region.

Embodiment 3 is the virus of Embodiment 1 or 2 wherein the at least one Mtb protein encoded by the second coding region is the same as the at least one Mtb protein encoded by the third coding region.

Embodiment 4 is the virus of any preceding Embodiment wherein the second coding region encodes a polycistronic message encoding at least two Mtb proteins.

Embodiment 5 is the virus of any preceding Embodiment wherein the third coding region encodes a polycistronic message encoding at least two Mtb proteins.

Embodiment 6 is the virus of any preceding Embodiment wherein the Mtb proteins encoded by the second coding region are different than the Mtb proteins encoded by the third coding region.

Embodiment 7 is the virus of any preceding Embodiment wherein the Mtb proteins encoded by the second coding region are the same as the Mtb proteins encoded by the third coding region.

Embodiment 8 is the virus of any preceding Embodiment wherein the second coding region expresses a monocistronic message encoding at least two Mtb proteins, wherein the monocistronic message comprises nucleotides encoding a self-cleaving peptide, and wherein the nucleotides are located between the Mtb proteins.

Embodiment 9 is the virus of any preceding Embodiment wherein the third coding region expresses a monocistronic message encoding at least two Mtb proteins, wherein the monocistronic message comprises nucleotides encoding a self-cleaving peptide, and wherein the nucleotides are located between the Mtb proteins.

Embodiment 10 is the virus of any preceding Embodiment wherein the self-cleaving peptide comprises a 2A peptide.

Embodiment 11 is the virus of any preceding Embodiment wherein the 2A peptide comprises a P2A peptide, a T2A peptide, a E2A peptide, or a F2A peptide.

Embodiment 12 is the virus of any preceding Embodiment wherein the at least one Mtb protein of the second genomic segment and the at least one Mtb protein of the third genomic segment are selected from acute-phase infection proteins, latency phase proteins, and resuscitation proteins.

Embodiment 13 is the virus of any preceding Embodiment wherein the at least two Mtb proteins of the second genomic segment and the at least two Mtb proteins of the third genomic segment are selected from acute-phase infection proteins, latency phase proteins, and resuscitation proteins.

Embodiment 14 is the virus of any preceding Embodiment wherein the acute-phase infection proteins are selected from the group consisting of Ag85A, Ag85B, ESAT-6(EsxA), and EsxH.

Embodiment 15 is the virus of any preceding Embodiment wherein the latency phase proteins is Rv1733.

Embodiment 16 is the virus of any preceding Embodiment wherein the resuscitation proteins is RpfA.

Embodiment 17 is the virus of any preceding Embodiment wherein the second coding region or the third coding region encodes an Ag84B protein.

Embodiment 18 is the virus of any preceding Embodiment wherein the second coding region or the third coding region encodes a monocistronic message encoding an ESAT-6(EsxA) protein and an EsxH protein, wherein the monocistronic message comprises nucleotides encoding a self-cleaving peptide, and wherein the nucleotides are located between the nucleotides encoding the ESAT-6(EsxA) protein and the EsxH protein.

Embodiment 19 is the virus of any preceding Embodiment wherein the second coding region or the third coding region encodes a monocistronic message encoding an Ag85B protein and an EsxH protein, wherein the monocistronic message comprises nucleotides encoding a self-cleaving peptide, and wherein the nucleotides are located between the nucleotides encoding the Ag85B protein and the EsxH protein.

Embodiment 20 is the virus of any preceding Embodiment wherein the second coding region or the third coding region encodes a monocistronic message encoding a RpfA protein and a Rv1733 protein, wherein the monocistronic message comprises nucleotides encoding a self-cleaving peptide, and wherein the nucleotides are located between the nucleotides encoding the RpfA protein and the Rv1733 protein.

Embodiment 21 is an infectious virus particle comprising the three genomic segments of any preceding Embodiment.

Embodiment 22 is a composition comprising the isolated infectious virus particle of any preceding Embodiment.

Embodiment 23 is a collection of vectors comprising: a first vector encoding the first genomic segment of any preceding Embodiment, wherein the first genomic segment is antigenomic, a second vector encoding the second genomic segment of any preceding Embodiment, wherein the second genomic segment is antigenomic, and a third vector encoding the third genomic segment of any preceding Embodiment, wherein the third genomic segment is antigenomic.

Embodiment 24 is the collection of any preceding Embodiment wherein the vectors are plasmids.

Embodiment 25 is the collection of any preceding Embodiment wherein the plasmids further comprise a T7 promoter.

Embodiment 26 is a method for making a genetically engineered Pichinde virus comprising: introducing into a cell the collection of vectors of any preceding Embodiment; and incubating the cells in a medium under conditions suitable for expression and packaging of the first, second, and third genomic segments.

Embodiment 27 is the method of any preceding Embodiment further comprising isolating an infectious virus particle from the medium.

Embodiment 28 is the method of any preceding Embodiment wherein the cells express a T7 polymerase.

Embodiment 29 the isolated infectious virus particle produced by the method of any preceding Embodiment.

Embodiment 30 a composition comprising the isolated infectious virus particle of any preceding Embodiment.

Embodiment 31 is a reverse genetics system for making a genetically engineered virus comprising three vectors, wherein a first vector encodes the first genomic segment of any preceding Embodiment, wherein the first genomic segment is antigenomic, wherein the second vector encodes the second genomic segment of any preceding Embodiment, wherein the second genomic segment is antigenomic, and wherein the third vector encodes the third genomic segment of any preceding Embodiment, wherein the third genomic segment is antigenomic.

Embodiment 32 is the reverse genetics system of any preceding Embodiment wherein each plasmid comprises a T7 promoter.

Embodiment 33 is a method for using a reverse genetics system, comprising: introducing into a cell the three vectors of genomic segments of any preceding Embodiment; and incubating the cell under conditions suitable for the transcription of the three genomic segments and expression of the coding regions of each genomic segment.

Embodiment 34 is the method of any preceding Embodiment further comprising isolating infectious virus particles produced by the cell, wherein each infectious virus particle comprises the three genomic segments.

Embodiment 35 the method of any preceding Embodiment wherein the introducing comprises transfecting a cell with the three genomic segments.

Embodiment 36 is the method of any preceding Embodiment wherein the introducing comprises contacting the cell with an infectious virus particle comprising the three genomic segments.

Embodiment 37 is the method of any preceding Embodiment wherein the cell is ex vivo.

Embodiment 38 is the method of any preceding Embodiment wherein the cell is a vertebrate cell.

Embodiment 39 is the method of any preceding Embodiment wherein the vertebrate cell is a mammalian cell.

Embodiment 40 is the method of any preceding Embodiment wherein the mammalian cell is a human cell.

Embodiment 41 is the method of any preceding Embodiment wherein the vertebrate cell is an avian cell.

Embodiment 42 is the method of any preceding Embodiment wherein the avian cell is a chicken embryonic fibroblast.

Embodiment 43 is a method for producing an immune response in a subject, comprising administering to a subject the infectious virus particle of any preceding Embodiment.

Embodiment 44 is the method of any preceding Embodiment wherein the subject is a vertebrate.

Embodiment 45 is the method of any preceding Embodiment wherein the vertebrate is a mammal.

Embodiment 46 is the method of any preceding Embodiment wherein the mammalian is a human.

Embodiment 47 is the method of any preceding Embodiment wherein the immune response comprises a humoral immune response.

Embodiment 48 is the method of any preceding Embodiment wherein the immune response comprises a cell-mediated immune response.

### EXAMPLES

The present disclosure is illustrated by the following examples. It is to be understood that the particular examples, materials, amounts, and procedures are to be interpreted broadly in accordance with the scope and spirit of the disclosure as set forth herein.

### Example 1

### Recombinant Pichinde Viral Vector Expressing Tuberculosis Antigens Elicits Strong T Cell Responses and Protection in Mice

Tuberculosis (TB) caused by Mycobacterium tuberculosis (Mtb) remains a major global health threat. The only available vaccine Bacille Calmette-Guérin (BCG) does not prevent adult pulmonary TB. New effective TB vaccines should aim to stimulate robust T cell responses in the lung mucosa to achieve high protective efficacy. Using a tri-segmented Pichinde virus (PICV) vector (rP18tri), viral vectored TB vaccines (TBvac-1, TBvac-2, and TBvac-10) encoding several known TB immunogens (Ag85B, EsxH, and ESAT-6/EsxA) have been generated. A P2A linker sequence was used to allow for the expression of two proteins from one open-reading-frame (ORF) on the viral RNA segments. The immunogenicity of TBvac-2 and TBvac-10 was evaluated in mice. Both viral vectored vaccines elicited strong antigen-specific CD4 and CD8 T cells through intramuscular (IM) and intranasal (IN) routes as evaluated by MHC-I and MHC-II tetramer analyses, respectively. The IN inoculation route helped to elicit strong lung T cell responses. The vaccine-induced antigen-specific memory CD4 T cells are functional, expressing multiple cytokines as detected by intracellular cytokine staining. Finally, immunization with TBvac-1 or TBvac-2, both expressing the same trivalent antigens (Ag85B, EsxH, ESAT6/EsxA), reduced Mtb lung tissue burden and dissemination in an aerosol challenge mouse model. In summary, the novel PICV vector-based TB vaccine candidates can express more than two antigens via the use of P2A linker sequence and elicit strong systemic and lung T cell immunity with protective efficacy.

### Introduction

Control of Mtb infection is generally believed to require cellular immune responses(7,8). CD4 Th1 cells can activate phagocytes by producing the IFNgamma and TNFalpha cytokines and cytotoxic CD8 T cells can kill Mtb-infected cells(9). In TB vaccine-immunized non-human primates (NHPs), T cell subsets associated with protection include multifunctional antigen-specific Th1/Th17 CD4 and CD8 T cells(10,11), and Mtb-specific CD4 and CD8 T cells with circulating central memory or tissue-resident memory phenotypes(12). Therefore, new TB vaccines should aim to stimulate robust cellular immunity in the lung mucosa to achieve high protective efficacy.

Viral vector vaccines mimic natural infection and elicit robust cell-mediated and humoral immune responses without the need of adjuvant (13). Many viral vectored vaccines have been approved for veterinary diseases (13), and several were approved for human usage in recent years. A Zaire ebolavirus vaccine based on a recombinant vesicular stomatitis virus vector (ERVEBO®) was approved by the Food and Drug Administration (FDA) in 2019(14). COVID-19 vaccines based on adenovirus vectors, such as the adenovirus type 26 (Ad26)-based Janssen vaccine and the Chimpanzee adenovirus vector (ChAd)-based Oxford/AstraZeneca vaccine, have been used during the pandemic under emergency use authorizations from the FDA(15) or the World Health Organization (WHO)(16,17). Viral vectors have also been explored for TB vaccine development. A rhesus CMV vector multivalent vaccine (RhCMV/TB) reduced Mtb infection and disease in NHPs(12), but the strict species specificity of CMVs prevents evaluation of RhCMV in other animal models and in humans. Phase 1 clinical trials have shown that two viral vectors, human adenovirus 5 (hAd5)(19) and MVA(20), are safe and elicit both systemic and mucosal immunity in trial participants. These studies set the stage for further development of mucosal vaccines targeting the respiratory system.

Described here is a novel viral vaccine vector based on a tri-segmented recombinant Pichinde virus (PICV) rP18tri, which can accommodate two additional open-reading frames (ORFs)(21,22). PICV is an arenavirus first isolated from rice rats in Colombia and is not known to cause human disease(23). The rP18tri vector is attenuated both in vitro and in vivo and causes a self-limiting infection in animals without any adverse effects or shedding of detectable virus in blood or body fluids(21). For these reasons, rP18tri has an increased safety profile compared to other live viral vectors. The low seroprevalence of PICV in human populations(23) also mitigates concern over pre-existing anti-vector immunity that is known for some widely used viral vectors, including hAd5(24). The rP18tri vector induces even stronger immune responses upon a homologous boost and can be repeatedly administered without significantly decreasing the vaccine immunity(21). The live-attenuated rP18tri vector elicits strong adaptive immunity(21), possibly because PICV targets antigen-presenting cells early in the infection, which enhances antigen presentation and increases the frequency and avidity of specific adaptive immunity. rP18tri can be given to many mammalian host species through various routes such as intramuscular (IM), intraperitoneal (IP), intranasal (IN), and oral(21).

This example provides a proof-of-concept that rP18tri-based multivalent TB vaccine candidates stimulate robust systemic and lung T cell immune responses with protective efficacy in mice. Three viral vectored vaccines TBvac-1, TBvac-2, and TBvac-10 expressing multiple Mtb antigens including Ag85B, EsxH, and ESAT-6/EsxA were generated. The quantity, phenotype, and functionality of T cells elicited by these viral vectored vaccines was characterized, and protection in a virulent Mtb challenge mouse model was evaluated. The results suggest the PICV vector as an attractive vaccine platform for use of Ag85B, EsxH, and ESAT6/EsxA as effective TB vaccine candidates.

### Materials and methods

### Mammalian Cells, Viruses, and Mtb Strain

BHK21 Baby hamster kidney cells and Vero African green monkey kidney cells were grown in DMEM media (Fisher Scientific) with 10% fetal bovine serum (FBS) (Sigma) and 50 micrograms (g) per milliliter (ml) penicillin and streptomycin (Invitrogen-LifeTechnologies). BSRT7-5 cells, obtained from K.K. Conzelmann (Ludwig-Maximilians-Universitat, Germany), are BHK-21 cells stably expressing T7 RNA polymerase. BSRT7-5 cells were grown in minimal essential medium (MEM) (Invitrogen-LifeTechnologies) with 10% FBS, 1 μg/ml Geneticin (Invitrogen-LifeTechnologies), and 50 μg/ml penicillin-streptomycin. Recombinant PICV-vectored TB vaccines were amplified in BHK-21 cells. Infectious virus titer was determined by viral plaque assay in Vero cells as described previously (25). Mycobacterium tuberculosis Erdman is a fully virulent isolate and a BSL-3 agent. Mtb was routinely cultured at 37° C. with aeration in Middlebrook 7H9 liquid medium (Difco), which was supplemented with 10% albumin-dextrose-saline (ADS), 0.5% glycerol and 0.1% Tween-80.

### Proteins, Peptides, Tetramers, and Antibodies

Mtb proteins, peptide arrays, and antibodies were obtained from BET Resources, NIAID, NIH. These include purified Ag85B (NR-14857) and ESAT-6/EsxA (NR-49424) proteins from Mtb strain H37Rv, peptide arrays of Mtb Ag85B (NR-34828) and ESAT-6 (NR-50711), rabbit polyclonal anti-ESAT6 (NR-13803) and anti-Ag85 Complex (NR-13800) antisera. Phycoerythrin (PE)-conjugated EsxH MHC-I tetramer (H-2K(b) IMYNYPAM), PE-conjugated MHC-II control and Ag85B tetramers were obtained from the NIH Tetramer Core Facility at Emory University. Antibodies used for T cell analysis of immunized mice were purchased from commercial sources, including allophycocyanin (APC)-labeled anti-CD3 (17A2) (Biolegend), fluorescein isothiocyanate (FITC)-labeled anti-CD4 (Biolegend), peridinin chlorophyll protein (PerCP)-Cy5.5-labeled anti-CD8 (53-6.7) (eBioscience), brilliant violent (BV) 510-labelled anti-CD44 (Biolegend), APC-Cy7 anti-CD69 (Biolegend), and BV421 anti-CD103 (BD Biosciences).

Generation of rP18Tri-Based TB Vaccines

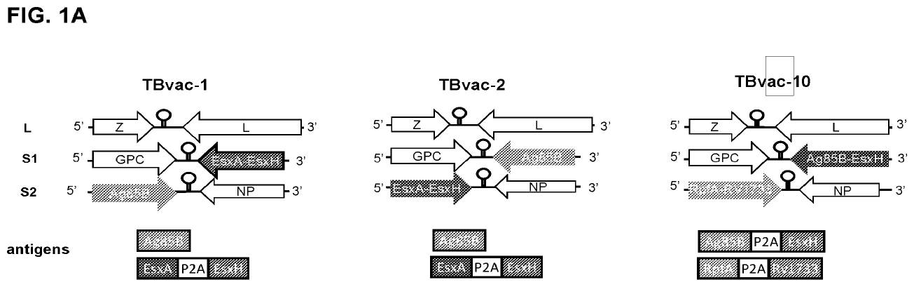

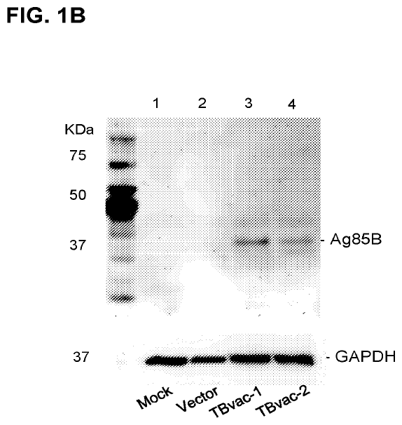

Recombinant tri-segmented PICV (rP18tri)-based TB vaccines were constructed as described previously(21,22). Sequences of Mtb antigens Ag85B (NP_216402), EsxH/TB10.4 (NP_214802), ESAT6/EsxA (YP_178023), Rv1733c (NP 216249), and RpfA (NP 215382) were obtained from GenBank. Gene fragments encoding Ag85B single gene and dual antigens (EsxA-EsxH, Ag85B-EsxH, and RpfA-Rv1733c) linked by a P2A linker sequence (GSGATNFSLLKQAGDVEENPGP, SEQ ID NO:9)(25) were codon optimized for human cell expression and chemically synthesized by Genewiz (Azenta Life Sciences). These gene fragments were cloned into the S1 or S2 vector of the rP18tri reverse genetics system between KpnI and XhoI sites(21,22). The resulting plasmids were verified by restriction enzyme digestion and sequencing. To rescue recombinant rP18tri vector-based TB vaccines, BSRT7-5 cells seeded at 1.5×10⁵ cells per ml in a 6-well plate were transfected with three plasmids expressing the L segment, the S1 and S2 segments encoding respective antigens as illustrated in FIG. 1A, using Lipofectamine™ 3000 Transfection Reagent (Thermo Fisher Scientific, L3000015). From 2 to 4 days post-transfection, the supernatants were collected for plaque assay on Vero cells. Individual plaques were picked to infect BHK-21 cells on 10-cm plates for 48 h to prepare viral stocks. Viral RNA was extracted from viral stocks using the QIAamp Viral RNA kit (Qiagen, USA), subjected to RT-PCR and sequencing to confirm the identity of viral vectored TB vaccines. TBvac-1 encodes EsxA-EsxH on the S1 segment and Ag85B on the S2 segment, while TBvac-2 encodes Ag85B on the S1 segment and EsxA-EsxH on the S2 segment. TBvac-10 encodes Ag85B-EsxH on the S1 segment and RpfA-Rv1733c on the S2 segment.

Plaque Assay to Quantify Viral Titer of rP18Tri-Based TB Vaccines

Vero cells in six-well plates were washed and infected with 0.5 ml of viruses in 10-fold serial dilutions. After incubation for 1 h at 37° C., the infection medium was removed. Fresh MEM supplemented with 0.5% agar and 10% FBS were added to the cells, which were cultured for 4 days at 37° C. Plaques were stained overnight with neutral red solution diluted (1:50) in 0.5% agar-MEM-10% FBS.

### Detection of Antigen Expression in TBvac-Infected Cells

Vero cells grown on coverslips were mock infected or infected with the rP18tri vectors alone or respective TBvac viruses at MOI of 0.1. At 24 hours post infection (hpi), cells were fixed with 4% paraformaldehyde for 15 min at room temperature, and washed three times with phosphate buffer saline (PBS) (Invitrogen-LifeTechnologies). Cells were treated with 0.1% Triton X-100 for 12 min followed by incubation with rabbit polyclonal antibody against Ag85B or ESAT6 (BEI) for 1 h. After washing, cells were incubated with goat anti-rabbit Alexa Fluor-488 (Invitrogen) for 1 hr at room temperature. The cells were washed three times and mounted on a glass slide for examination by confocal microscopy.

### Mouse Immunization and Tissue Collection

For immunogenicity studies, female six- to eight-week-old C57BL/6 mice were obtained from Jackson Laboratories and housed for at least one week for acclimatization. Groups of three to five mice were immunized intranasally (IN) or intramuscularly (IM) depending on the study protocol with 1×10⁵ PFU of TBvac-2, TBvac-10, rP18tri vector, or phosphate buffered saline (PBS) as a mock control. To evaluate immune responses after primary immunization, blood was collected into lithium heparin tubes (Greiner Bio-One) via the facial vein. Peripheral blood mononuclear cells (PBMCs) were isolated by first lysing red blood cells (RBC) using 1×RBC lysis buffer (eBiosciences) and then washing in PBS with 2% fetal bovine serum (FBS) (Sigma Aldrich). Three weeks after primary immunization, mice were boosted IN or IM with the same vaccine candidate. Seven days after the boost, mice were euthanized by CO₂ inhalation and the spleen and lung collected into sterile PBS. To isolate splenocytes, spleens were gently crushed through a 40 m cell strainer and washed with RPMI-1640 medium with L-glutamine (Cytiva HyClone). Red blood cells were lysed using 1×RBC lysis buffer and splenocytes resuspended in PBS with 2% FBS. To isolate lung lymphocytes, lungs were cut into small pieces and incubated for 1 hour at 37 C in PBS+5% FBS with 1.5 mg/ml type I collagenase (need manufacturer) and 2 g/ml DNase I (New England BioLabs). Then, lungs were gently crushed through a 40 m cell strainer, washed with RPMI1640/1% FBS and 10 mM HEPES (ThermoFisher). Cells were resuspended in 44% Percoll (GE Healthcare), underlain with 67% Percoll, centrifuged, and the lymphocyte layer collected. Lymphocytes were resuspended in PBS with 2% FBS.

### IFNgamma-Based ELISPOT Assay

ELISPOT was performed using Mouse IFNγ ELISpot Development Module (R&D Systems, SEL485) following the manufacturer's instruction. Briefly, 96-well PVDF-bottom Immunospot plates were coated with anti-mouse IFNγ capture antibody overnight at 4° C. The next day, plates were washed and blocked in 1% bovine serum albumin (BSA)/5% sucrose/PBS. 1×10⁵ splenocytes were added per well and incubated with medium only or with peptide pools of Ag85B or ESAT6/EsxA (2 g/ml) (BEI) for 18 hours at 37° C. and 5% CO2. After incubation, biotinylated anti-mouse IFNγ detection antibody was added and incubated overnight at 4° C. The following day, streptavidin-alkaline phosphatase and BCIP/NBT were added for color development. Spots were recorded using CTL ImmunoSpot Reader and quantified using immunospot Fluox suite software.

### Evaluation of Antigen-Specific CD8 T Cells by MHC-I Tetramer Analysis

The PE-labeled MHC-I tetramer H-2K(b)/EsxH epitope IMYNYPAM was provided by the NIH Tetramer core facility at Emory University (Atlanta, GA). Isolated PBMCs, splenocytes, and lung lymphocytes were incubated with PE-labeled EsxH/H-2K(b) MHC-II tetramer, fixable viability stain 780 (BD Biosciences), APC-labeled anti-CD3 (Biolegend), PerCP-Cy5.5-labeled anti-CD8 (eBioscience), FITC-labeled anti-CD4 (Biolegend), and BV510-labeled anti-CD44 (Biolegend) for one hour at room temperature. Cells were washed three times with PBS/2% PBS. Sample acquisition was performed on FACSCelesta and data analyzed using FlowJo.

### Evaluation of Antigen-Specific CD4 T Cells by MHC-II Tetramer Analysis

The PE-labeled MHC-II Ag85B tetramer I-A(b)/FQDAYNAAGGHNAVF and control tetramer I-A(b)/PVSKMRMATPLLMQA were provided by the NIH Tetramer core facility at Emory University (Atlanta, GA). Isolated PBMCs, splenocytes, and lung lymphocytes were incubated with fixable viability stain 780 (BD Biosciences), APC-labeled anti-CD3 (Biolegend), PerCP-Cy5.5-labeled anti-CD8 (eBioscience), FITC-labeled anti-CD4 (Biolegend), and BV510-labeled anti-CD44 (Biolegend), together with either control or Ag85B PE-labeled MHC-II tetramer. To detect tissue-resident CD4 T cells in the lung, lung lymphocytes were incubated with the same cocktails, together with APC-Cy7-labeled anti-CD69 (Biolegend). Cells were washed three times with PBS/2% PBS. Sample acquisition was performed on FACSCelesta and data analyzed using FlowJo.

### Intracellular Cytokine Staining

Isolated cells were seeded in leukocyte medium (complete RPMI) at 1×10⁶ cells per well and incubated with medium alone or with purified Ag85B/ESAT-6 proteins (each 10 g/ml) obtained from BEI for 2 hours at 37° C. Cells were further incubated for six hours at 37° C. with anti-CD28 antibody and GolgiStop (BD Biosciences). Cells were collected, washed with PBS/2% FBS, and stained with fixable viability dye 780, PerCP-Cy5.5-labeled anti-CD3, FITC-labeled anti-CD8, APC-Cy7-labeled anti-CD4, and PE-Cy7-labeled anti-CD44 for 45 minutes on ice. Cells were then fixed and permeabilized using the Cytofix/Cytoperm kit (BD Biosciences) and stained with BV421-labeled anti-TNFα, PE-labeled anti-IL-2, and APC-labeled anti-IFNγ for 40 minutes on ice. Sample events acquisition was performed on FACSCelesta and data analyzed using FlowJo.

### Mth Mouse Aerosol Challenge Model

Female C57BL/6 mice of six-to-eight weeks old were immunized IM with 1×10⁵ PFU of rP18tri vector, TBvac-1, or TBvac-2 in a prime-boost strategy with a 21-day interval. At 28 days after immunization, vaccinated mice were challenged with ~400 CFU of virulent Mtb Erdman, using a Glas-Col Inhalation Exposure System, in a biosafety level 3 (BSL3) laboratory, as previously described (26). A group of unvaccinated mice (n=4) were euthanized at 24 hr post-infection to determine the Mtb dose. At 4- and 12-weeks post-infection, groups of mice (n=4-5) were sacrificed; lungs and spleen were collected for bacterial quantification. Tissues were homogenized (BioGen Pro200) in 3 ml of PBS containing 0.05% Tween-80 (PBS-T), serially diluted in PBS-T and plated on Middlebrook 7H10 agar (Difco) supplemented with 0.5% glycerol, 10% oleic acid-albumin-dextrose-catalase (OADC, Difco) and 100 μg/ml cycloheximide. Plates were incubated for 3-4 weeks at 37° C. before counting CFU.

### Results

Generation of rP18Tri Viral Vector Vaccines Encoding Mtb Antigens

The genome of wild-type PICV consists of two RNA segments, each encoding two genes in opposite orientations(27). The L segment encodes a matrix protein Z and a large RNA polymerase L, while the S segment encodes the envelope glycoprotein GPC and the nucleoprotein NP(28). By splitting the S segment into the S1 and S2 segments, we were able to insert an ORF of up to 2 kilobases in each segment (FIG. 1A)(21,22).

Two immunodominant Mtb antigens EsxH/TB10.4 and Ag85B were used as they have well-established major histocompatibility complex (MHC) class I (MHC-I) and MHC-II tetramer assays to conveniently evaluate the antigen-specific CD8 and CD4 T cells, respectively, in mice(29-33). To evaluate whether multiple antigens expressed from the same vector would interfere with the immune development, additional antigens ESAT-6/EsxA, Rv1733c, and RpfA were included that are associated with different stages of Mtb life cycle and have been included in other vaccine design (12). To maximize the coding capacity of an ORF, we used a P2A linker between two antigens. The P2A linker sequence is a self-cleaving peptide that introduces ribosomal skipping during translation, allowing the production of multiple proteins from one ORF (26, 34). We aim to test whether effective immune responses are induced to each antigen linked by the P2A sequence. This may represent a valid strategy to generate rP18tri-based multivalent vaccines (33,35,36).

Codon-optimized genes were cloned into either the S1 or S2 vector as illustrated in FIG. 1A. Recombinant rP18tri-based TB vaccines, TBvac-1, TBvac-2, and TBvac-10, were generated using reverse genetics as described previously (21,22,37). TBvac-1 and TBvac-2 encode the same Mtb antigens, Ag85B and EsxA-EsxH, except that the antigen location is swapped between the Si and S2 segments (FIG. 1A). TBvac-10 encodes Ag85B-EsxH within the Si ORF, and RpfA-Rv1733c within the S2 ORF (FIG. 1A). In this study, we used TBvac-2 and TBvac-10 to characterize T cell immune responses and evaluated TBvac-1 and TBvac-2 for protective efficacy against virulent Mtb challenge.

To detect antigen expression from viral vectored vaccines, Vero cells were mock infected or infected with rP18tri vector or viral vectored TB vaccines at MOI of 0.1 for 24 h. Ag85B expression was detected in cells infected with vaccines but not with vector or mock infection by both Western blotting (FIG. 1B) and immunofluorescence assay (IFA). EsxA expression in cells infected with TBvac-2 was detected, but not with mock, vector, or TBvac-10 by IFA. It was not possible to assess EsxH, RpfA or Rv1733c expression due to lack of antibody reagents.

### TBvac-2 Induces Functional Antigen-Specific T Cell Responses by ELISpot Assay

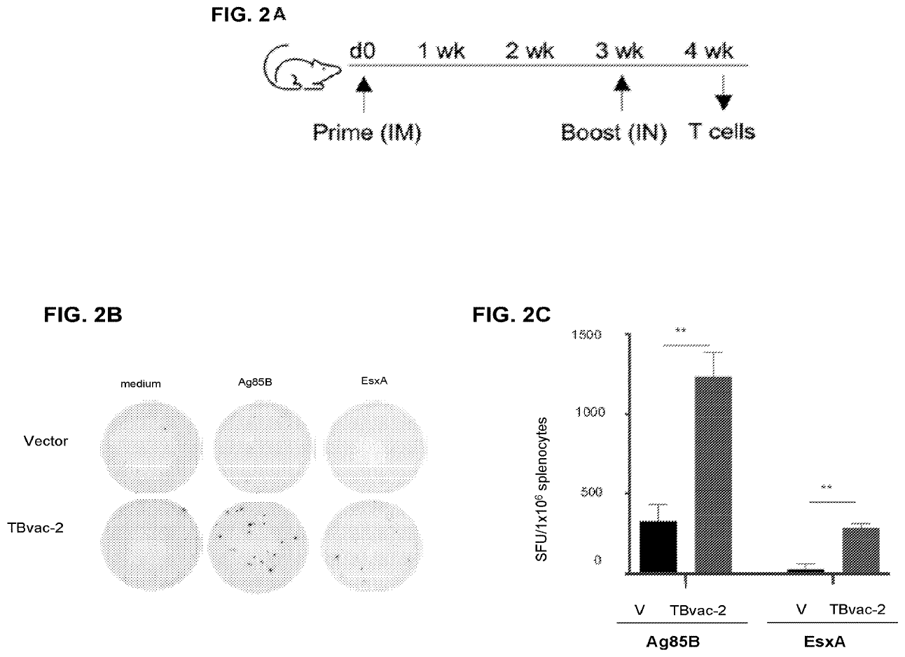

Vaccine-induced T cell responses were first evaluated by IFNgamma-based enzyme-linked immunosorbent spot (ELISpot) assay, which detects production of IFNgamma from lymphocytes after antigen stimulation. The rP18tri vector-based influenza vaccine induces even stronger antibody and T cell responses upon boost than after prime(21), so T cell responses were evaluated only after the boost dose. C57BL/6J (BL6) mice (n=3) were immunized with either the rP18tri vector (V) or TBvac-2 by an IM-IN prime-boost strategy (FIG. 2A). At 7 days post-boost (dpb), splenocytes were analyzed by mouse IFNgamma-based ELISpot assay after stimulation with medium alone or respective peptide pools (FIG. 2B). After normalization to the medium control, the number of IFNgamma-secreting cells after Ag85B or ESAT-6/EsxA peptide stimulation was significantly increased in TBvac-2 immunized compared to vector immunized mice (FIG. 2C). These findings show that immunization with TBvac-2 elicits IFNgamma-producing, antigen-specific T cell responses.

### TBvac-2 and TBvac-10 Induce Strong Antigen-Specific Memory CD8 T Cells Through Both IM and IN Routes

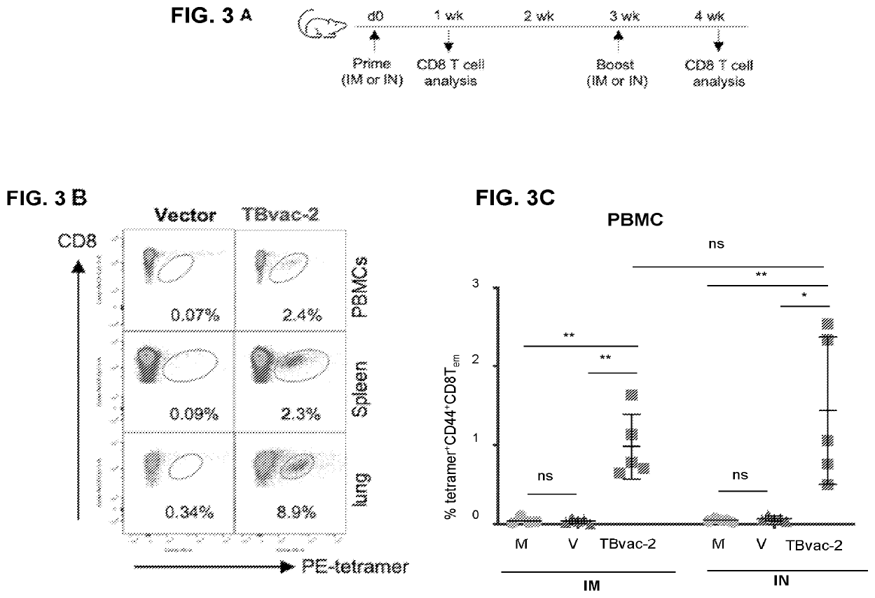

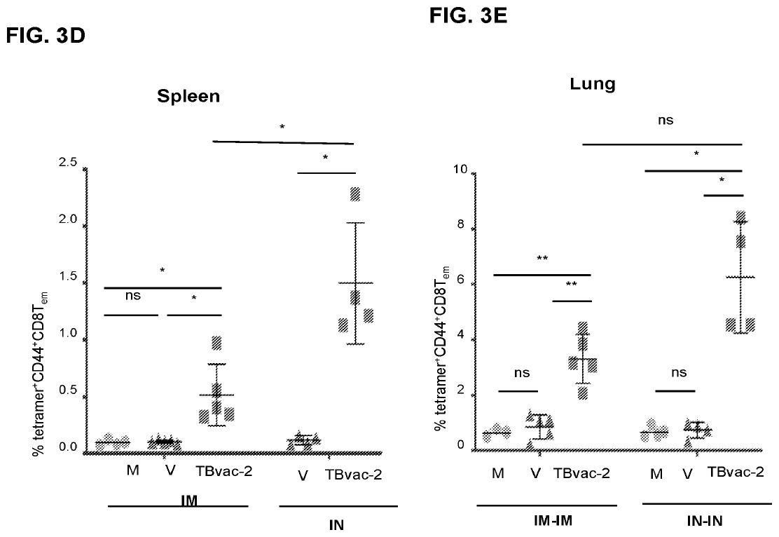

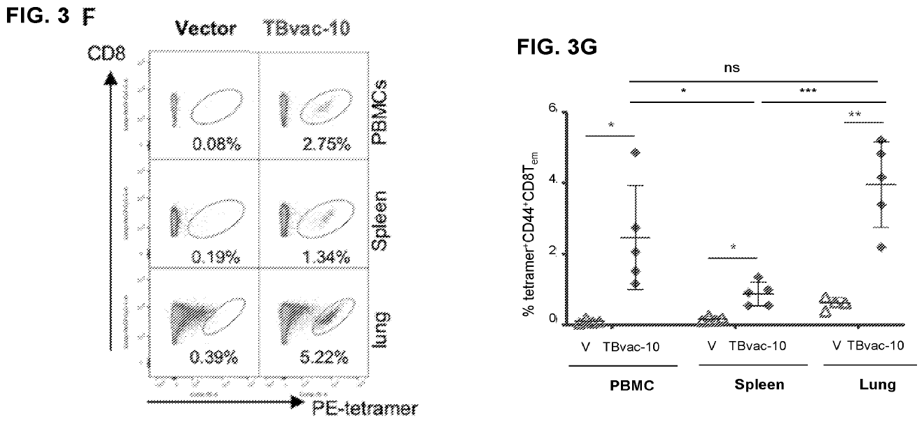

The vaccine-induced antigen-specific CD8 T cell responses after either systemic (IM) or mucosal (IN) inoculation were evaluated using an established MHC-I EsxH tetramer assay. BL6 mice were immunized with PBS/mock, rP18tri vector alone, or TBvac-2 through either IM or IN route (FIG. 3A). At 7 d post-prime (dpp), lymphocytes from peripheral blood (PBMC), spleen, and lung were analyzed by EsxH-specific MHC-I tetramer analysis (FIG. 3B). A single dose immunization of TBvac-2 through either IM or IN route generated a significantly higher percentage of MHC-I EsxH tetramer-positive CD8 T cells in the peripheral blood (FIG. 3C) and spleen (FIG. 3D) than mock or vector immunization. No significant difference in the PBMC CD8 T cell response was detected between the IM and IN routes (FIG. 3C), while IN appears to elicit higher CD8 T cell responses than IM in the spleen (FIG. 3D). The tetramer-positive CD8 T cells in the lung were also quantified at 7 d post-boost after prime-boost immunization by the IM-IM or IN-IN routes (FIG. 3A). Consistent with the results observed in PBMCs and spleen, TBvac-2 immunization via both routes generated a significantly higher percentage of tetramer-positive CD8 T cells in the lung than mock or vector alone, with a trend toward higher responses in mice immunized by the IN route compared to IM, though this did not achieve statistical significance (FIG. 3E).

As both routes of inoculation effectively stimulated immunity, we elected to move forward with a heterologous prime-boost strategy (IM-IN), which has been shown to result in greater protection against M/b in guinea pigs(38). CD8 T cell responses induced in mice immunized IM-IN with TBvac-10 were evaluated by the MHC-I EsxH tetramer assay (FIG. 3F). Mice immunized with TBvac-10 had a significantly higher percentage of tetramer-positive CD8 T cells compared to vector immunized animals in the peripheral blood, spleen, and lung (FIG. 3G). The percentage of antigen-specific CD8 T cells was significantly higher in the lung than in the spleen.

Taken together, these results show that two different rP18tri-based TB vaccines (TBvac-2 and TBvac-10) administered systemically, mucosally, or a combination of the two, elicit strong antigen-specific CD8 T cells in the spleen, peripheral blood, and lungs. As EsxH is expressed after the P2A linker in both TBvac-2 and TBvac-10, these results also demonstrate that P2A-linked antigens in the rP18tri viral vector can stimulate robust immune responses.

### TBvac-2 and TBvac-10 Induce Functional Antigen-Specific CD4 T Cells in the Lungs

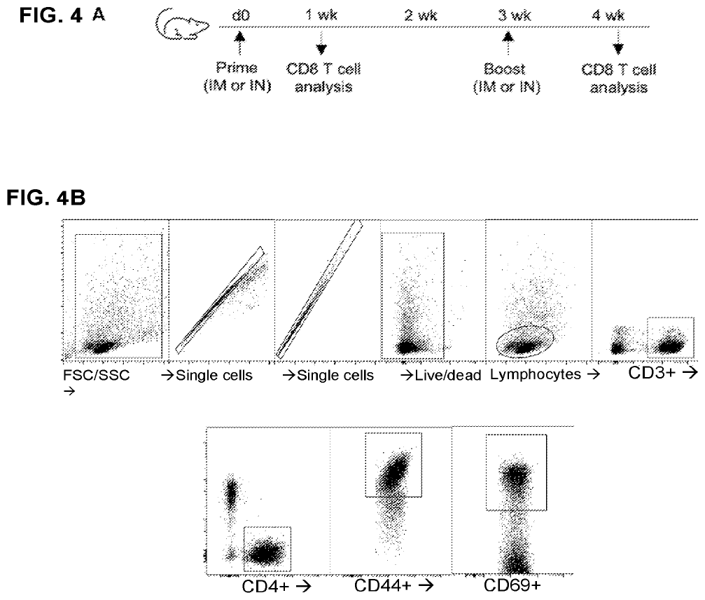

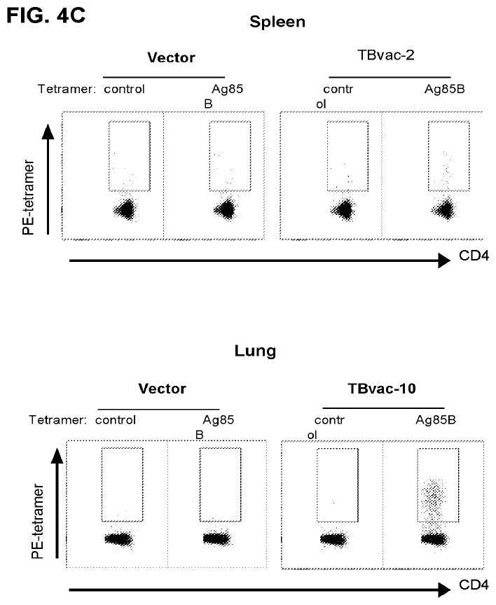

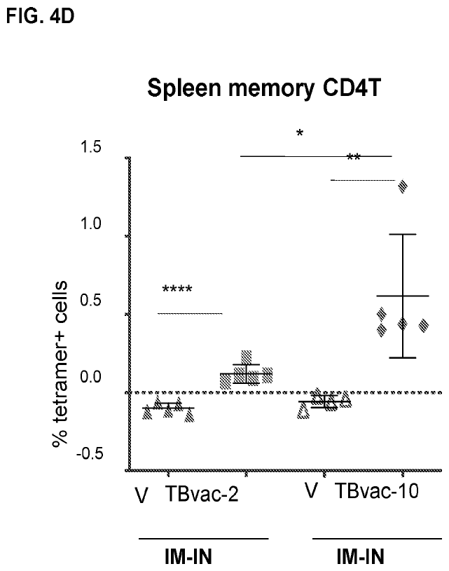

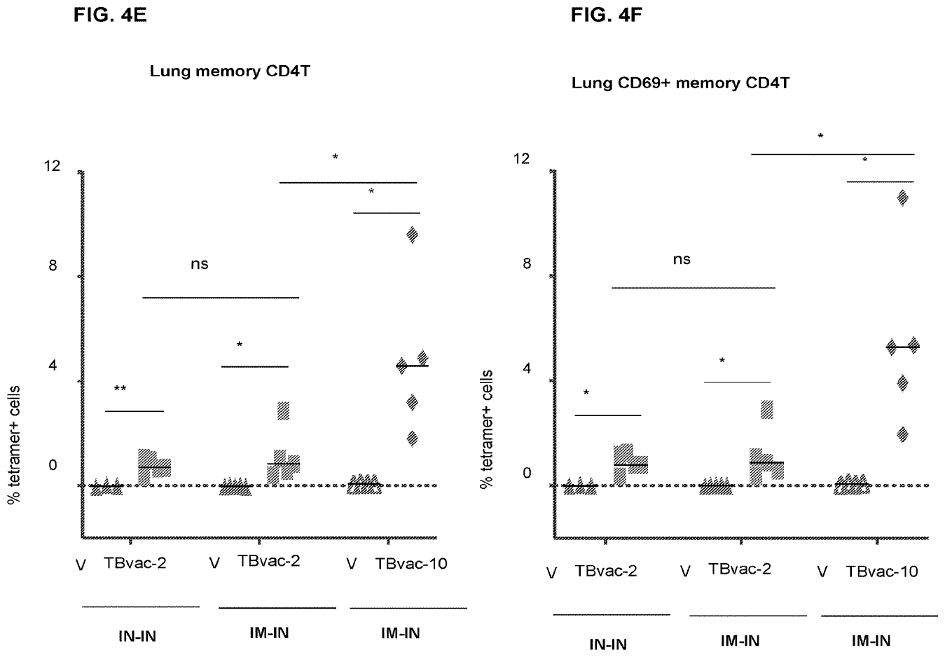

The vaccine-induced CD4 T cell responses by MHC-II tetramer analysis were evaluated. BL6 mice were prime-boosted with vector (V), TBvac-2, or TBvac-10 by either the IM-IN or IN-IN routes. Spleen and lung lymphocytes were analyzed for Ag85B MHC-II tetramer-positive CD4 T cells (FIG. 4B). A gating strategy for analyzing the flow cytometry data is shown in FIG. 4B. Both spleen and lung lymphocytes were gated for CD3⁺CD4⁺CD44⁺ population and lung lymphocytes were further gated by CD69⁺, which is a tissue-resident cell surface marker(39). Tetramer-positive cells in the target cell populations were specifically detected in TBvac-2 and TBvac-10-immunized mice after incubation with Ag85B MHC-II tetramer (FIG. 4C). After normalization with the control MHC-II tetramer, the percentage of Ag85B MHC-II tetramer-positive cells among CD4 T cells in the spleen (FIG. 4D) and lung (FIG. 4E) were determined for each mouse. In both tissues, significantly higher percentages of tetramer-positive cells were detected in mice immunized with TBvac-2 and TBvac-10 than with the vector, demonstrating the induction of antigen-specific CD4 T cells by both vaccines. TBvac-10 induced a higher percentage of tetramer-positive CD4 T cells in both the spleen (FIG. 4D) and lung (FIG. 4E) compared to TBvac-2, suggesting a stronger Ag85B-specific CD4 T cell immunity. No significant differences in lung tetramer-positive CD4 T cells were detected between IN-IN and IM-IN routes of inoculation with TBvac-2 (FIG. 4E).

Recent studies suggest that tissue-resident memory T cells (Trm) play an important role in the primary defense against infection (40) and that Mtb-specific CD4 and CD8 T cells with tissue-resident memory phenotypes are associated with protection (12). To determine whether the vaccine-induced lung CD4 T cells exhibit tissue-resident phenotypes, we gated the CD69⁺ (a tissue-resident cell marker) population of the CD4 T cells (CD3⁻CD4⁺CD44⁺CD69⁺) for quantification of tetramer-positive cells. Both TBvac-2 and TBvac-10 stimulated high levels of tetramer-positive CD69⁺ CD4 T cells in the lung (FIG. 4F). Consistent with the results for CD4 T cells (FIGS. 4D and 4E), TBvac-10 immunization induced a higher level of CD69⁺CD4 T cells than TBvac-2 (FIG. 4F).

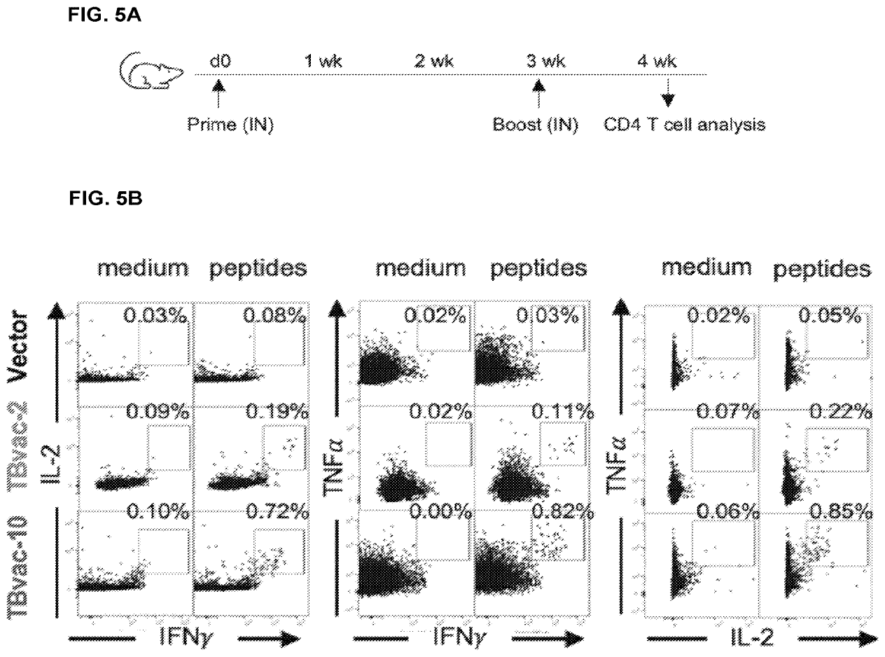

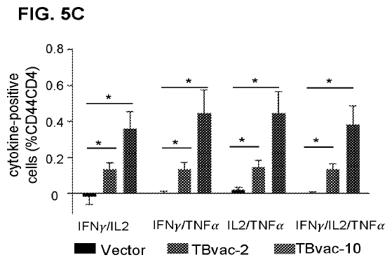

As multifunctional T cells are associated with control of multiple intracellular pathogens(41,42) including Mtb (43), we also evaluated whether the viral vaccines can induce antigen-specific functional CD4 T cells by intracellular cytokine staining after ex vivo stimulation with antigen peptide pools (FIG. 5A). CD4 T cells that were positive for dual cytokine expression (IFNgamma/IL-2, IFNgamma/TNFalpha, and IL-2/TNFalpha) were specifically detected after peptide stimulation in TBvac-2 and TBvac-10 immunized mice but not in vector-immunized mice (FIG. 5B). The average percentages of dual- and triple-positive (IFNgamma/IL-2/TNFalpha) memory CD4 T cells were significantly higher in mice receiving either vaccine than in the vector group (FIG. 5C). There was a trend toward greater percentage of multifunctional cells for TBvac-10 than TBvac-2, but this was not statistically significant.

Taken together, these results suggest that both TBvac-2 and TBvac-10 can elicit functional antigen-specific CD4 T cells systemically and locally (lung) and that TBvac-10 may induce a stronger CD4 T cell immunity than TBvac-2.

rP18tri-based TB vaccines reduced bacterial load in mouse lungs in a virulent Mtb infection model

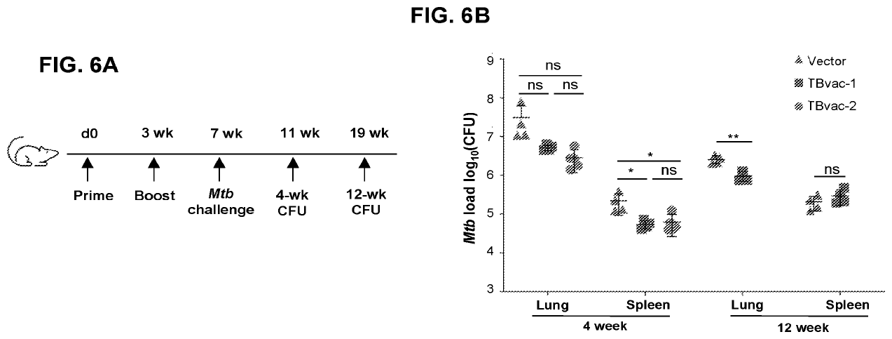

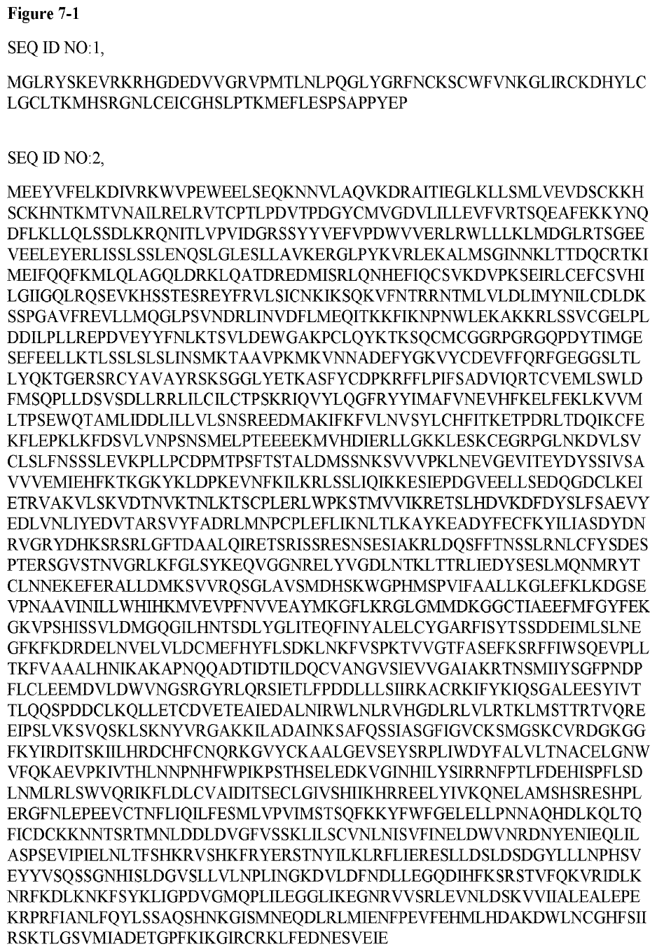

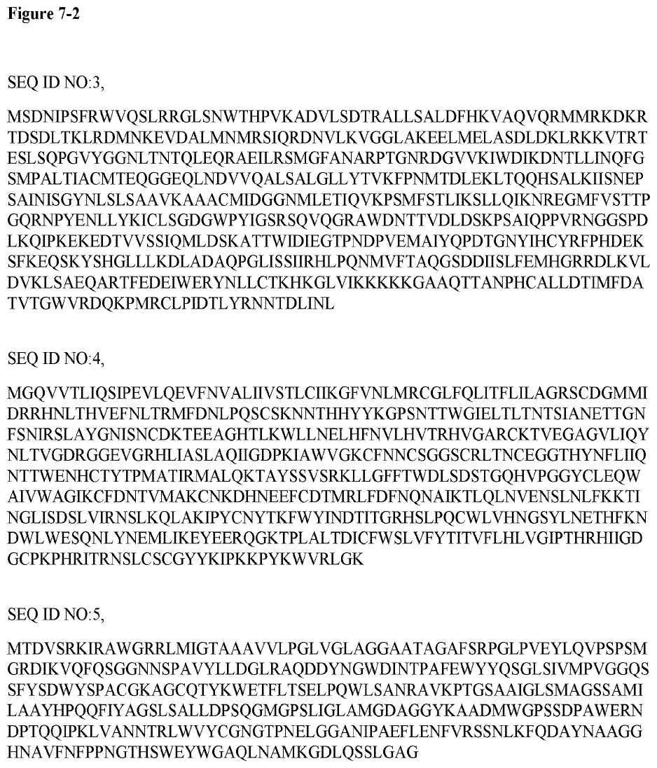

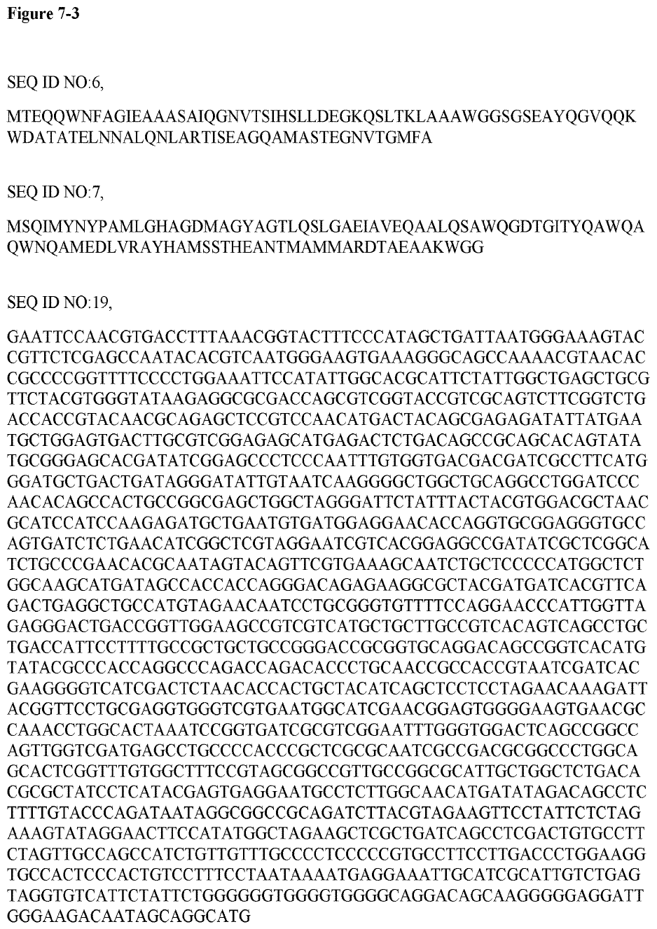

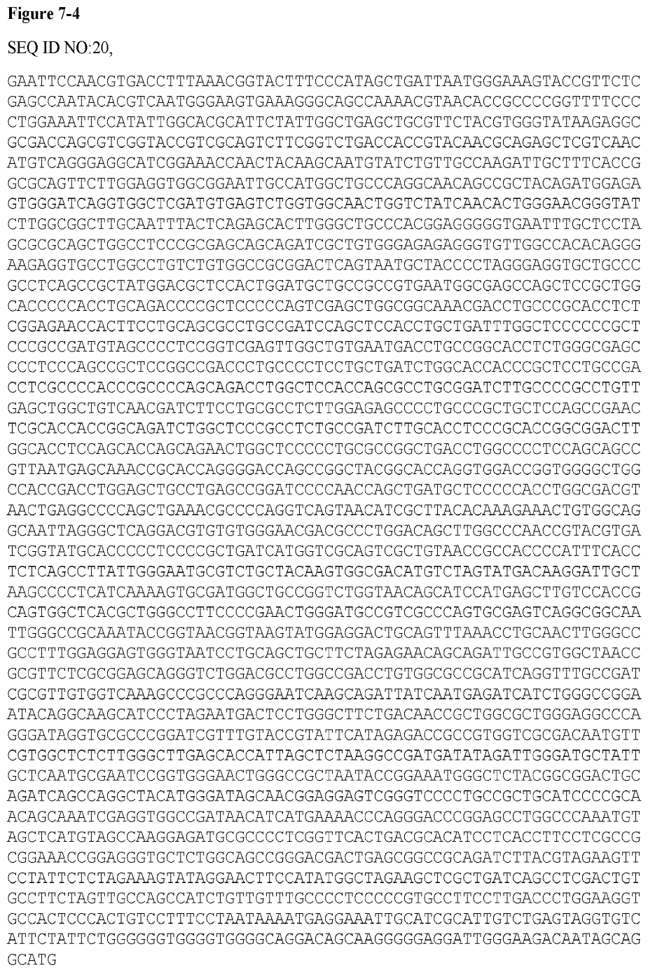

An initial study to evaluate the protective efficacy of TBvac-1 and TBvac-2 in a standard Mtb low-dose aerosol challenge mouse model (44) was conducted. BL6 mice prime-boost immunized through the IM route with 1×10⁵ PFU of vector alone, TBvac-1, or TBvac-2 were infected by aerosol with virulent Mtb Erdman and euthanized at 4 or 12 weeks post-infection for bacterial load quantification (FIG. 6A). At the 4-week timepoint, TBvac-1 and TBvac-2-immunized mice showed a similarly decreased bacterial load in both lungs and spleens (FIG. 6B). The average Mtb burden in the lungs of TBvac-1 and TBvac-2-immunized mice decreased by 0.8 to 1 log compared to vector-immunized mice (FIG. 6B). Although not statistically significant, this decrease is similar to what is seen in BL6 mice immunized with BCG alone (45, 46). TBvac-1 and TBvac-2 immunization also significantly lowered the bacterial load in the spleens at the 4-week time point compared to vector alone (FIG. 6B). At the 12-week time point, we included the vector and TBvac-1 groups, but not TBvac-2 group, due to insufficient number of animals. TBvac-1 immunized animals had significantly decreased Mtb loads in the lung but not in the spleen (FIG. 6B). These results establish that rP18tri-based vaccines expressing the Mtb antigens Ag85B, EsxA, and EsxH reduce the bacterial burden in mice challenged with virulent Mtb, demonstrating their potential for further development.

### Discussion

An expanded pipeline of TB vaccine candidates is needed to ensure development of a highly effective TB vaccine. The goal of this study was to evaluate the immunogenicity and efficacy of novel multiantigen PICV-vectored TB vaccine candidates in mice. We show that immunization with these vaccine candidates elicits high levels of antigen-specific CD4 and CD8 T cells in the peripheral blood, spleen, and lung. These antigen-specific T cells are functional in secreting multiple cytokines (IFNgamma, IL-2, and TNFalpha) upon antigen stimulation and express tissue-resident markers in the lung. While CD4 T cells are traditionally thought to exert control over Mth(8), recent work has shown that vaccine-induced CD8 T cells mediate protection in mice(47,48). Thus, the PICV-vectored TB vaccine candidates have the potential to induce protective immunity by stimulating both arms of the T cell immune response. Indeed, we show that immunization with the PICV-vectored TB vaccines can reduce Mtb lung tissue burden and dissemination in an aerosol challenge mouse model.

A major advantage of the PICV vector is its multiple delivery routes including systemic and mucosal sites that provide various options to efficiently control different pathogens. TB is a respiratory disease. Though IM vaccination is the most common route of immunization in people, recent studies indicate that mucosal immunization with TB vaccines results in robust local immunity(19,20) and may be more protective(48,49). Systemic (IM) and mucosal (IN) routes of the PICV-vectored vaccines in this study were compared. Consistent with our previous findings (21), both routes of inoculation can elicit strong antigen-specific CD8 T cells after a single dose. The IN route seemed to elicit a higher CD8 T cell response than the IM route, both locally in the lung and systemically in spleen and blood, though the difference was not always statistically significant. We further showed that prime-boost via the IM-IN routes induced a similarly high level of CD4 T cells in the lung as via the IN-IN routes, suggesting that a mucosal boost immunization of the PICV-vectored vaccine is sufficient to induce effective lung T cell immunity. Our results are consistent with a published study showing that a systemic IM prime and IN boost of a viral vector vaccine elicited efficient T cell homing to the lungs(50).

A mucosal immunization of the live PICV vector is expected to elicit Trm cells. Upon IN inoculation, the PICV-vectored vaccines preferentially targets antigen-presenting cells (APCs) (13), which migrate to the mediastinal lymph node, where effector memory T cells can be activated, migrate into the lung tissue, and convert into Trm(51). Trm cells are located at barrier epithelial sites and rapidly respond to pathogens via cytotoxicity and cytokine production(39,52). A protective role for Trm during Mtb infection has been shown in NHPs(49) and mice(53). Trm-mediated protection against Mtb in mice occurs following immunization with either mucosal BCG (48) or a recombinant influenza A virus-vectored vaccine(54). Functional Trm have also been identified in the respiratory tracts of people with active TB disease(55). The important role of Trm in Mtb control supports the development of mucosal TB vaccines designed to elicit Trm(56). Multiple viral vectored mucosal TB vaccines have been shown to effectively stimulate CD4 and/or CD8 Trm after respiratory immunization (19,47,54). We show here that the PICV-vectored vaccine candidates after the IN inoculation can elicit a high level of lung CD4 T cells expressing tissue-resident marker CD69. Whether these are Trm cells remains to be validated in future studies using intravascular staining and expanded Trm cell markers such as CD103 and CD11a.

The three PICV-vectored vaccine candidates used in this study encode two common antigens EsxH and Ag85B. TBvac-10 elicits more Ag85B-specific circulating and lung tissue-resident CD4 T cells than TBvac-2, possibly due to an increased antigen production and/or presentation. Ag85B is expressed alone for TBvac-2, but linked to EsxH through a P2A linker for TBvac-10. These differences may affect when, where, and how much Ag85B is expressed in viral vaccine-infected cells, leading to differential magnitude of T cell responses. Nevertheless, studies with TBvac-10 suggest that antigens linked by P2A sequences can each induce strong T cell responses. Thus, inclusion of multiple antigens utilizing the two ORFs within the PICV vector is feasible. The Mtb antigens tested in this study represent a small subset of T cell targets. They were selected for convenient quantification of T cell responses but these antigens such as EsxA and Ag85B are suboptimal for inducing protective immunity(32). Incorporation of new immunogens in the PICV vector platform, using the strategy validated in this study, has the potential to increase protection.

We demonstrated that systemic (IM) immunization of PICV-based TB vaccine candidates could partially protect mice from virulent Mtb infection in an aerosol infection model. Two candidates (TBvac-1 and TBvac-2) expressing the same three Mtb antigens (Ag85B, EsxH, and ESAT-6/EsxA), decreased bacterial load in the lung and the spleen by ~1 log at the 4-week time point in Mtb-infected mice compared to the viral vector alone. TBvac-1 (TBvac-2 not tested) also reduced the bacterial load in the lung but not in the spleen at the 12-week time point. This might be explained by the delayed Mtb dissemination from the lung to the spleen, or less durable protection in the spleen than in the lung. Future studies to analyze the long-lived memory cells at both lung and spleen infection sites will help address this question. A relatively high dose (~400 CFU) of virulent Mtb was used in the challenge model, which might have obscured some protective effect of the vaccines. As a mock-immunized group was not included in the protection experiment, we could not determine whether general immune stimulation induced by the PICV vector could further contribute to protection. Such vector-induced protection has been documented with other viral vectors (57) and will be important to evaluate in further studies. Nevertheless, the antigen-specific immunity elicited by TBvac-1 and TBvac-2 was able to decrease the bacterial load by ~1 log compared to the vector group, similar to the level of protection induced by BCG vaccination in the low-dose aerosol mouse model (45, 46).

In summary, we have shown that novel PICV-based TB vaccines elicit strong systemic and lung T cell immunity, and confer protection against Mtb infection in a virulent mouse model. Our study supports the continued development of multiantigen TB vaccine candidates using the PICV vector platform.

### CITATIONS

1. World Health Organization (WHO). Global tuberculosis report 2022. www.who.int/publications-detail-redirect/9789240061729 [Accessed Nov. 1, 2022]
2. Houben R M G J, Dodd P J. The Global Burden of Latent Tuberculosis Infection: A Re-estimation Using Mathematical Modelling. PLoS Med (2016) 13:e1002152. doi: 10.1371/journal.pmed.1002152
3. WHO. Global Tuberculosis Report 2021. https://www.who.int/teams/global-tuberculosis-programme/tb-reports/global-tuberculosis-report-2021 [Accessed Feb. 6, 2022]
4. Andersen P, Kaufmann S H E. Novel vaccination strategies against tuberculosis. Cold Spring Harb Perspect Med (2014) 4: doi: 10.1101/cshperspect.a018523
5. Mangtani P, Abubakar I, Ariti C, Beynon R, Pimpin L, Fine P E M, Rodrigues L C, Smith P G, Lipman M, Whiting P F, et al. Protection by BCG vaccine against tuberculosis: a systematic review of randomized controlled trials. Clin Infect Dis (2014) 58:470-480. doi: 10.1093/cid/cit790
6. Tait D R, Hatherill M, Van Der Meeren O, Ginsberg A M, Van Brakel E, Salaun B, Scriba T J, Akite E J, Ayles H M, Bollaerts A, et al. Final Analysis of a Trial of M72/AS01E Vaccine to Prevent Tuberculosis. N Engl J Med (2019) 381:2429-2439. doi: 10.1056/NEJMoa1909953
7. Orme I M, Collins F M. Protection against Mycobacterium tuberculosis infection by adoptive immunotherapy. Requirement for T cell-deficient recipients. J Exp Med (1983) 158:74-83. doi: 10.1084/jem.158.1.74
8. Mogues T, Goodrich M E, Ryan L, LaCourse R, North R J. The relative importance of T cell subsets in immunity and immunopathology of airborne Mycobacterium tuberculosis infection in mice. J Exp Med (2001) 193:271-280. doi: 10.1084/jem.193.3.271
9. O'Garra A, Redford P S, McNab F W, Bloom C I, Wilkinson R J, Berry M P R. The immune response in tuberculosis. Annu Rev Immunol (2013) 31:475-527. doi: 10.1146/annurev-immunol-032712-095939
10. Darrah P A, Zeppa J J, Maiello P, Hackney J A, Wadsworth M H, Hughes T K, Pokkali S, Swanson P A, Grant N L, Rodgers M A, et al. Prevention of tuberculosis in macaques after intravenous BCG immunization. Nature (2020) 577:95-102. doi: 10.1038/s41586-019-1817-8
11. Dijkman K, Sombroek C C, Vervenne R A W, Hofman S O, Boot C, Remarque E J, Kocken C H M, Ottenhoff T H M, Kondova I, Khayum M A, et al. Prevention of tuberculosis infection and disease by local BCG in repeatedly exposed rhesus macaques. Nat Med (2019) 25:255-262. doi: 10.1038/s41591-018-0319-9
12. Hansen S G, Zak D E, Xu G, Ford J C, Marshall E E, Malouli D, Gilbride R M, Hughes C M, Ventura A B, Ainslie E, et al. Prevention of tuberculosis in rhesus macaques by a cytomegalovirus-based vaccine. Nat Med (2018) 24:130-143. doi: 10.1038/nm.4473
13. Vrba SM, Kirk NM, Brisse ME, Liang Y, Ly H. Development and Applications of Viral Vectored Vaccines to Combat Zoonotic and Emerging Public Health Threats. Vaccines (Basel) (2020) 8: doi: 10.3390/vaccines8040680
14. Commissioner O of the. First FDA-approved vaccine for the prevention of Ebola virus disease, marking a critical milestone in public health preparedness and response. FDA (2020) https://www.fda.gov/news-events/press-announcements/first-fda-approved-vaccine-prevention-ebola-virus-disease-marking-critical-milestone-public-health [Accessed Nov. 29, 2022]
15. Commissioner O of the. Janssen COVID-19 Vaccine. FDA (2022) https://www.fda.gov/emergency-preparedness-and-response/coronavirus-disease-2019-covid-19/janssen-covid-19-vaccine [Accessed Nov. 29, 2022]
16. World Health Organization (WHO). Interim recommendations for use of the ChAdOx1-S [recombinant] vaccine against COVID-19 (AstraZeneca COVID-19 vaccine AZD1222 Vaxzevria™, SII COVISHIELD™). https://www.who.int/publications-detail-redirect/WHO-2019-nCoV-vaccines-SAGE_recommendation-AZD1222-2021.1 [Accessed Nov. 3, 2022]
17. WHO. Interim recommendations for use of the ChAdOx1-S [recombinant] vaccine against COVID-19 (AstraZeneca COVID-19 vaccine AZD1222 Vaxzevria™, SII COVISHIELD™). https://www.who.int/publications-detail-redirect/WHO-2019-nCoV-vaccines-SAGE_recommendation-AZD1222-2021.1 [Accessed Nov. 29, 2022]
18. Vierboom M P M, Chenine A L, Darrah P A, Vervenne R A W, Boot C, Hofman S O, Sombroek C C, Dijkman K, Khayum M A, Stammes M A, et al. Evaluation of heterologous prime-boost vaccination strategies using chimpanzee adenovirus and modified vaccinia virus for TB subunit vaccination in rhesus macaques. NPJ Vaccines (2020) 5:39. doi: 10.1038/s41541-020-0189-2
19. Jeyanathan M, Fritz D K, Afkhami S, Aguirre E, Howie K J, Zganiacz A, Dvorkin-Gheva A, Thompson M R, Silver R F, Cusack R P, et al. Aerosol delivery, but not intramuscular injection, of adenovirus-vectored tuberculosis vaccine induces respiratory-mucosal immunity in humans. JCI Insight (2022) 7:e155655. doi: 10.1172/jci.insight.155655
20. Satti I, Meyer J, Harris S A, Manjaly Thomas Z-R, Griffiths K, Antrobus R D, Rowland R, Ramon R L, Smith M, Sheehan S, et al. Safety and immunogenicity of a candidate tuberculosis vaccine MVA85A delivered by aerosol in BCG-vaccinated healthy adults: a phase 1, double-blind, randomised controlled trial. Lancet Infect Dis (2014) 14:939-946. doi: 10.1016/S1473-3099(14)70845-X
21. Dhanwani R, Zhou Y, Huang Q, Verma V, Dileepan M, Ly H, Liang Y. A Novel Live Pichinde Virus-Based Vaccine Vector Induces Enhanced Humoral and Cellular Immunity after a Booster Dose. J Virol (2016) 90:2551-2560. doi: 10.1128/JVI.02705-15
22. Dhanwani R, Ly H, Liang Y. Recombinant Tri-Segmented Pichinde Virus as a Novel Live Viral Vaccine Platform. Methods Mol Biol (2017) 1581:169-179. doi: 10.1007/978-1-4939-6869-5_10
23. Trapido H, Sanmartin C. Pichinde virus, a new virus of the Tacaribe group from Colombia. Am J Trop Med Hyg (1971) 20:631-641.
24. Coughlan L. Factors Which Contribute to the Immunogenicity of Non-replicating Adenoviral Vectored Vaccines. Front Immunol (2020) 11:909. doi: 10.3389/fimmu.2020.00909
25. Donnelly M L L, Luke G, Mehrotra A, Li X, Hughes L E, Gani D, Ryan M D. Analysis of the aphthovirus 2A/2B polyprotein “cleavage” mechanism indicates not a proteolytic reaction, but a novel translational effect: a putative ribosomal “skip.” J Gen Virol (2001) 82:1013-1025. doi: 10.1099/0022-1317-82-5-1013
26. Ramakrishnan P, Aagesen A M, McKinney J D, Tischler A D. Mycobacterium tuberculosis Resists Stress by Regulating PE19 Expression. Infect Immun (2015) 84:735-746. doi: 10.1128/IAI.00942-15
27. Lan S, McLay Schelde L, Wang J, Kumar N, Ly H, Liang Y. Development of infectious clones for virulent and avirulent pichinde viruses: a model virus to study arenavirus-induced hemorrhagic fevers. J Virol (2009) 83:6357-6362. doi: 10.1128/JVI.00019-09
28. Buchmeier M J, de la Torre J-C, Peters CJ. “Arenaviridae.,” Fields Virology.

Philadelphia: Wolters Kluwer Health/Lippincott Williams & Wilkins (2013). p. 1283-1303
29. Bertholet S, Ireton GC, Kahn M, Guderian J, Mohamath R, Stride N, Laughlin E M, Baldwin S L, Vedvick T S, Coler R N, et al. Identification of human T cell antigens for the development of vaccines against Mycobacterium tuberculosis. J Immunol (2008) 181:7948-7957. doi: 10.4049/jimmunol.181.11.7948
30. Lindestam Arlehamn C S, Gerasimova A, Mele F, Henderson R, Swann J, Greenbaum J A, Kim Y, Sidney J, James E A, Taplitz R, et al. Memory T cells in latent Mycobacterium tuberculosis infection are directed against three antigenic islands and largely contained in a CXCR3+CCR6+ Th1 subset. PLoS Pathog (2013) 9:e1003130. doi: 10.1371/journal.ppat.1003130
31. Orr M T, Ireton G C, Beebe E A, Huang P-W D, Reese V A, Argilla D, Coler R N, Reed S G. Immune subdominant antigens as vaccine candidates against Mycobacterium tuberculosis. J Immunol (2014) 193:2911-2918. doi: 10.4049/jimmunol.1401103
32. Moguche A O, Musvosvi M, Penn-Nicholson A, Plumlee CR, Mearns H, Geldenhuys H, Smit E, Abrahams D, Rozot V, Dintwe O, et al. Antigen Availability Shapes T Cell Differentiation and Function during Tuberculosis. Cell Host Microbe (2017) 21:695-706.e5. doi: 10.1016/j.chom.2017.05.012
33. Hoang T, Aagaard C, Dietrich J, Cassidy JP, Dolganov G, Schoolnik G K, Lundberg C V, Agger E M, Andersen P. ESAT-6 (EsxA) and TB10.4 (EsxH) based vaccines for pre- and post-exposure tuberculosis vaccination. PLoS One (2013) 8:e80579. doi: 10.1371/journal.pone.0080579
34. Aagaard C, Hoang T, Dietrich J, Cardona P-J, Izzo A, Dolganov G, Schoolnik G K, Cassidy J P, Billeskov R, Andersen P. A multistage tuberculosis vaccine that confers efficient protection before and after exposure. Nat Med (2011) 17:189-194. doi: 10.1038/nm.2285
35. Leung-Theung-Long S, Gouanvic M, Coupet C-A, Ray A, Tupin E, Silvestre N, Marchand J-B, Schmitt D, Hoffmann C, Klein M, et al. A Novel MVA-Based Multiphasic Vaccine for Prevention or Treatment of Tuberculosis Induces Broad and Multifunctional Cell-Mediated Immunity in Mice and Primates. PLoS One (2015) 10:e0143552. doi: 10.1371/journal.pone.0143552
36. Park A, Yun T, Hill T E, Ikegami T, Juelich T L, Smith J K, Zhang L, Freiberg A N, Lee B. Optimized P2A for reporter gene insertion into Nipah virus results in efficient ribosomal skipping and wild-type lethality. J Gen Virol (2016) 97:839-843. doi: 10.1099/jgv.0.000405
37. Dhanwani R, Huang Q, Lan S, Zhou Y, Shao J, Liang Y, Ly H. Establishment of Bisegmented and Trisegmented Reverse Genetics Systems to Generate Recombinant Pichindé Viruses. Methods Mol Biol (2018) 1604:247-253. doi: 10.1007/978-1-4939-6981-4_19
38. Xing Z, McFarland C T, Sallenave J-M, Izzo A, Wang J, McMurray D N. Intranasal mucosal boosting with an adenovirus-vectored vaccine markedly enhances the protection of BCG-primed guinea pigs against pulmonary tuberculosis. PLoS One (2009) 4:e5856. doi: 10.1371/journal.pone.0005856
39. Teijaro J R, Turner D, Pham Q, Wherry E J, Lefrangois L, Farber DL. Cutting edge: Tissue-retentive lung memory CD4 T cells mediate optimal protection to respiratory virus infection. J Immunol (2011) 187:5510-5514. doi: 10.4049/jimmunol.1102243
40. Masopust D, Soerens A G. Tissue-Resident T Cells and Other Resident Leukocytes. Annu Rev Immunol (2019) 37:521-546. doi: 10.1146/annurev-immunol-042617-053214
41. Darrah P A, Patel D T, De Luca P M, Lindsay R W B, Davey D F, Flynn B J, Hoff S T, Andersen P, Reed S G, Morris S L, et al. Multifunctional TH1 cells define a correlate of vaccine-mediated protection against Leishmania major. Nat Med (2007) 13:843-850. doi: 10.1038/nm1592
42. Kannanganat S, Kapogiannis B G, Ibegbu C, Chennareddi L, Goepfert P, Robinson H L, Lennox J, Amara R R. Human immunodeficiency virus type 1 controllers but not noncontrollers maintain CD4 T cells coexpressing three cytokines. J Virol (2007) 81:12071-12076. doi: 10.1128/JVI.01261-07
43. Lindenstrom T, Knudsen N P H, Agger E M, Andersen P. Control of chronic Mycobacterium tuberculosis infection by CD4 KLRG1-IL-2-secreting central memory cells. J Immunol (2013) 190:6311-6319. doi: 10.4049/jimmunol.1300248
44. Ordway D J, Orme I M. Animal models of mycobacteria infection. Curr Protoc Immunol (2011) Chapter 19:Unit19.5. doi: 10.1002/0471142735.im1905s94
45. Bertholet S, Ireton G C, Ordway D J, Windish H P, Pine S O, Kahn M, Phan T, Orme I M, Vedvick T S, Baldwin S L, et al. A defined tuberculosis vaccine candidate boosts BCG and protects against multidrug-resistant Mycobacterium tuberculosis. Sci Transl Med (2010) 2:53ra74. doi: 10.1126/scitranslmed.3001094
46. Lindenstrom T, Agger E M, Korsholm K S, Darrah P A, Aagaard C, Seder R A, Rosenkrands I, Andersen P. Tuberculosis subunit vaccination provides long-term protective immunity characterized by multifunctional CD4 memory T cells. J Immunol (2009) 182:8047-8055. doi: 10.4049/jimmunol.0801592
47. Hu Z, Wong K-W, Zhao H-M, Wen H-L, Ji P, Ma H, Wu K, Lu S-H, Li F, Li Z-M, et al. Sendai Virus Mucosal Vaccination Establishes Lung-Resident Memory CD8 T Cell Immunity and Boosts BCG-Primed Protection against TB in Mice. Mol Ther (2017) 25:1222-1233. doi: 10.1016/j.ymthe.2017.02.018
48. Perdomo C, Zedler U, Kihl A A, Lozza L, Saikali P, Sander L E, Vogelzang A, Kaufmann S H E, Kupz A. Mucosal BCG Vaccination Induces Protective Lung-Resident Memory T Cell Populations against Tuberculosis. mBio (2016) 7:e01686-16. doi: 10.1128/mBio.01686-16
49. Kaushal D, Foreman T W, Gautam U S, Alvarez X, Adekambi T, Rangel-Moreno J, Golden N A, Johnson A-M F, Phillips B L, Ahsan M H, et al. Mucosal vaccination with attenuated Mycobacterium tuberculosis induces strong central memory responses and protects against tuberculosis. Nat Commun (2015) 6:8533. doi: 10.1038/ncomms9533
50. Joag V, Wijeyesinghe S, Stolley J M, Quarnstrom C F, Dileepan T, Soerens A G, Sangala J A, O'Flanagan S D, Gavil N V, Hong S-W, et al. Cutting Edge: Mouse SARS-CoV-2 Epitope Reveals Infection and Vaccine-Elicited CD8 T Cell Responses. J Immunol (2021) 206:931-935. doi: 10.4049/jimmunol.2001400
51. Wu T, Hu Y, Lee Y-T, Bouchard K R, Benechet A, Khanna K, Cauley L S. Lung-resident memory CD8 T cells (TRM) are indispensable for optimal cross-protection against pulmonary virus infection. J Leukoc Biol (2014) 95:215-224. doi: 10.1189/jlb.0313180
52. Sakai S, Kauffman K D, Schenkel J M, McBerry C C, Mayer-Barber K D, Masopust D, Barber D L. Cutting edge: control of Mycobacterium tuberculosis infection by a subset of lung parenchyma-homing CD4 T cells. J Immunol (2014) 192:2965-2969. doi: 10.4049/jimmunol.1400019
53. Flórido M, Muflihah H, Lin L C W, Xia Y, Sierro F, Palendira M, Feng C G, Bertolino P, Stambas J, Triccas J A, et al. Pulmonary immunization with a recombinant influenza A virus vaccine induces lung-resident CD4+ memory T cells that are associated with protection against tuberculosis. Mucosal Immunol (2018) 11:1743-1752. doi: 10.1038/s41385-018-0065-9
54. Yang Q, Zhang M, Chen Q, Chen W, Wei C, Qiao K, Ye T, Deng G, Li J, Zhu J, et al. Cutting Edge: Characterization of Human Tissue-Resident Memory T Cells at Different Infection Sites in Patients with Tuberculosis. J Immunol (2020) 204:2331-2336. doi: 10.4049/jimmunol.1901326
55. Ogongo P, Porterfield J Z, Leslie A. Lung Tissue Resident Memory T-Cells in the Immune Response to Mycobacterium tuberculosis. Front Immunol (2019) 10:992. doi: 10.3389/fimmu.2019.00992
56. Zheng M Z M, Wakim L M. Tissue resident memory T cells in the respiratory tract. Mucosal Immunol (2022) 15:379-388. doi: 10.1038/s41385-021-00461-z
57. Beverley P C L, Ruzsics Z, Hey A, Hutchings C, Boos S, Bolinger B, Marchi E, O'Hara G, Klenerman P, Koszinowski U H, et al. A novel murine cytomegalovirus vaccine vector protects against Mycobacterium tuberculosis. J Immunol (2014) 193:2306-2316. doi: 10.4049/jimmunol.1302523

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A genetically engineered Pichinde virus comprising:
three ambisense genomic segments,
wherein the first genomic segment comprises a coding region encoding a Z protein and a coding region encoding a L RdRp protein,
wherein the second genomic segment comprises a coding region encoding a nucleoprotein (NP) and a second coding region, wherein the second coding region encodes at least one Mtb acute-phase protein, at least one Mtb latency phase protein, at least one Mtb protein expressed during the resuscitation phase, or a combination thereof, and
wherein the third genomic segment comprises a coding region encoding a glycoprotein and a third coding region, wherein the third coding region encodes at least one Mtb acute-phase protein, at least one Mtb latency phase protein, at least one Mtb protein expressed during the resuscitation phase, or a combination thereof.

2-3. (canceled)

4. The virus of claim 1 wherein the second coding region encodes a polycistronic message encoding at least two Mtb proteins.

5. The virus of claim 1 wherein the third coding region encodes a polycistronic message encoding at least two Mtb proteins.

6. The virus of claim 4 wherein the Mtb proteins encoded by the second coding region are different than the Mtb proteins encoded by the third coding region.

7. The virus of claim 4 wherein the Mtb proteins encoded by the second coding region are the same as the Mtb proteins encoded by the third coding region.

8. The virus of claim 1 wherein the second coding region expresses a monocistronic message encoding at least two Mtb proteins, wherein the monocistronic message comprises nucleotides encoding a self-cleaving peptide, and wherein the nucleotides are located between the Mtb proteins.

9. The virus of claim 1 wherein the third coding region expresses a monocistronic message encoding at least two Mtb proteins, wherein the monocistronic message comprises nucleotides encoding a self-cleaving peptide, and wherein the nucleotides are located between the Mtb proteins.

10. The virus of claim 8 wherein the self-cleaving peptide comprises a 2A peptide.

11-20. (canceled)

21. An infectious virus particle comprising the three genomic segments of claim 1.

22. A composition comprising the isolated infectious virus particle of claim 21.

23. A collection of vectors comprising:
a first vector encoding the first genomic segment of claim 1, wherein the first genomic segment is antigenomic,
a second vector encoding the second genomic segment of claim 1, wherein the second genomic segment is antigenomic, and
a third vector encoding the third genomic segment of claim 1, wherein the third genomic segment is antigenomic.

24-25. (canceled)

26. A method for making a genetically engineered Pichinde virus comprising:
introducing into a cell the collection of vectors of claim 23; and
incubating the cells in a medium under conditions suitable for expression and packaging of the first, second, and third genomic segments.

27-30. (canceled)

31. A reverse genetics system for making a genetically engineered virus comprising three vectors,
wherein a first vector encodes the first genomic segment of claim 1, wherein the first genomic segment is antigenomic,
wherein the second vector encodes the second genomic segment of claim 1, wherein the second genomic segment is antigenomic, and
wherein the third vector encodes the third genomic segment of claim 1, wherein the third genomic segment is antigenomic.

32. (canceled)

33. A method for using a reverse genetics system, comprising:
introducing into a cell the three vectors of genomic segments of claim 31; and
incubating the cell under conditions suitable for the transcription of the three genomic segments and expression of the coding regions of each genomic segment.

34-36. (canceled)

37. The method of claim 33 wherein the cell is ex vivo.

38. The method of claim 33 wherein the cell is a vertebrate cell.

39-40. (canceled)

41. The method of claim 38 wherein the vertebrate cell is an avian cell.

42. (canceled)

43. A method for producing an immune response in a subject, comprising
administering to a subject the infectious virus particle of claim 21.

44-45. (canceled)

46. The method of claim 43 wherein the subject is a human.

47. The method of claim 43 wherein the immune response comprises a humoral immune response, a cell-mediated immune response, or a combination thereof.

48. (canceled)
